(19) United States
(12) Patent Application Publication
(10) Pub. No.: US 2023/0265167 A1
(43) Pub. Date: Aug. 24, 2023

(54) COMPOSITIONS HAVING NEUROREGENERATIVE APPLICATIONS

(71) Applicant: GRIFOLS WORLDWIDE OPERATIONS LIMITED, DUBLIN (IE)

(72) Inventors: Thomas BARNETT, Research Triangle Park, NC (US); David A. ROSS, Research Triangle Park, NC (US)

(21) Appl. No.: 18/004,233

(22) PCT Filed: Jul. 7, 2021
(86) PCT No.: PCT/EP2021/068800

§ 371 Date: Jan. 4, 2023

Related U.S. Application Data
(60) Provisional application No. 63/049,516, filed on Jul. 8, 2020.

(51) Int. Cl. C07K 14/79; A61K 45/06; A61P 25/28; A61K 38/18

(52) U.S. Cl. CPC C07K 14/79; A61K 45/06; A61P 25/28; A61K 38/185

(57) ABSTRACT

Pharmaceutical compositions containing transferrin and, or lactoferrin for use in promoting and or inducing the generation new neural cells in a patient that has suffered a neurodegenerative event arising from at least one of a traumatic brain injury, a non-traumatic brain injury, a spinal cord injury, a peripheral nerve injury, or peripheral neuropathy. Ideally, the transferrin and/or lactoferrin have a low iron saturation.

### FIELD OF THE INVENTION

The present invention relates to therapeutic proteins and their use in the field of regenerative medicine. In particular, disclosed herein are applications of transferrin and lactoferrin and their use in promoting the proliferation, induction, and/or differentiation of neural progenitor cells or neural stem cells in patients that have suffered neural injuries.

### BACKGROUND

Injuries to the brain and spinal cord can manifest in immediate or chronic neurodegenerative effects that overwhelmingly alter the quality of the affected individual's life. Acquired brain injuries (ABI), i.e. injuries that are not hereditary, congenital, or induced by birth result in a changes to the brain's neuronal activity that affect the physical integrity, metabolic activity, or functional ability of nerve cells in the brain. There are two types of acquired brain injury: traumatic and non-traumatic.

Traumatic brain injuries (TBI) are characterised by an alteration in brain function, or other evidence of brain pathology, caused by an external force. Traumatic impact injuries can be defined as non-penetrating or penetrating, and include falls, assaults, motor vehicle accidents and sports injuries. Non-Traumatic Brain Injuries (NBTI) cause damage to the brain through non-impact processes such as a lack of oxygen, exposure to toxins, pressure from a tumour, etc. Examples of NTBI include stroke, aneurysms and lack of oxygen supply to the brain caused by heart attacks.

Stroke is one of the most prevalent categories of NBTI and occurs when the blood supply to part of the brain is interrupted or reduced, preventing brain tissue from getting oxygen and nutrients. Neurodegenerative processes commence almost immediately, and brain cells begin to die in minutes. An ischemic stroke occurs when a blood vessel (artery) supplying blood to an area of the brain becomes blocked by a blood clot. A haemorrhagic stroke happens when an artery in the brain leaks or ruptures. Of the two, ischemic stroke is the most common form of stroke.

The pervasiveness of stroke in modern society produces an immense burden on the health care infrastructure and spend. At present, recombinant tissue plasminogen activator (rtPA) is the only FDA-approved therapeutic agent for ischemic stroke. The major functions of rtPA are dissolving blood clots and promoting reperfusion. An alternative method for re-establishing a blocked blood flow is by a surgical intervention.

rtPA treatment has a narrow therapeutic time-window and as such its widespread applicability is limited. Moreover, although restoring perfusion via rtPA to the ischemic tissue is important, cascades of necrosis, apoptosis, and inflammation commence within minutes of severe oxygen deprivation. Increasingly, it is posited that genetically programmed neural cell death during post-ischemic tissue inflammation (which can last days to weeks) contributes significantly to the ultimate pathology because of delays in patient treatment/evaluation. As such, permanent neural and neuronal damage can arise before a patient even presents for evaluation.

At the time of writing, there are no approved neuroregenerative treatments that can reverse the effects of the neurodegeneration associated with TBI and NTBI. Given the lack of curative therapies it is not surprising that most of the literature in this field is biased towards neuroprotection; a non-restorative approach for mitigating neural cell death by prior or co-administering a particular molecule in response to an anticipated neurodegenerative ischemic, or reperfusion event.

One such example is U.S. Patent Publication No. 2016008437 in the name of Grifols Worldwide Operations Ltd which discloses apo-transferrin as exerting a neuroprotective effect by modulating the activity of Hypoxia Inducible Factors (HIF) in a rat model of stroke. The inventors observed a neuroprotective effect for apo-transferrin manifesting in a decrease in the volume of the infarcted area in the rats treated with apo-transferrin when compared with control rats.

Notwithstanding the foregoing it is immediately apparent that there is a paucity of clinical candidates having the potential to reverse the debilitating effects of neurodegeneration associated with spinal cord injury, TBI and NBTI. Therapies focussing on this need, and at least partially reversing traumatic and non-traumatic neural damage remain elusive and, as such, are highly desirable.

### DESCRIPTION OF THE INVENTION

The words “comprises/comprising” and the words “having/including” when used herein with reference to the present invention are used to specify the presence of stated features, integers, steps or components but do not preclude the presence or addition of one or more other features, integers, steps, components, or groups thereof.

It should be appreciated by those skilled in the art that the specific embodiments disclosed herein should not be read in isolation, and that the present specification intends for the disclosed embodiments to be read in combination with one another as opposed to individually. As such, each embodiment may serve as a basis for modifying or limiting other embodiments disclosed herein.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “10 to 100” should be interpreted to include not only the explicitly recited values of 10 to 100, but also include individual value and sub-ranges within the indicated range. Thus, included in this numerical range are individual values such as 10, 11, 12, 13 . . . 97, 98, 99, 100 and sub-ranges such as from 10 to 40, from 25 to 40 and 50 to 60, etc. This same principle applies to ranges reciting only one numerical value, such as “at least 10”. Furthermore, such an interpretation should apply regardless of the breadth of the range or the characteristics being described.

### Methods of Treatment

In a first aspect, the present invention provides for a method of promoting and or inducing the generation of new neural cells in a patient that has suffered a neurodegenerative event arising from at least one of a traumatic brain injury, a non-traumatic brain injury, a spinal cord injury, a peripheral nerve injury, or peripheral neuropathy,

the method comprising administering a therapeutically effective amount of a protein selected from transferrin, lactoferrin, and combinations thereof to the patient in need thereof.

In one embodiment, the patient may have suffered a neurodegenerative event arising from at least one of a stroke, a peripheral nerve injury, a traumatic brain injury, or peripheral neuropathy. For example, the patient may have suffered a neurodegenerative event arising from a peripheral nerve injury. In one embodiment, the patient may have suffered a neurodegenerative event arising from a stroke, such as an ischemic stroke or a haemorrhagic stroke. In one embodiment, the patient may have suffered a neurodegenerative event arising from an ischemic stroke.

The skilled person will appreciate that among the plethora of mammalian iron-binding proteins transferrin and lactoferrin are related proteins of the transferrin family with 61% sequence identity. In addition to a number of overlapping and complimentary functions, transferrin and lactoferrin also demonstrate a number of mutually exclusive functions. The present invention includes within its scope all wild type mammalian transferrin proteins, however, human transferrin (UniProtKB Seq. No. Q06AH7) comprising the amino acid sequence set forth in SEQ ID NO: 1 is particularly preferred. Similarly, the present invention includes within its scope all wild type mammalian lactoferrin proteins, however, human lactoferrin (UniProtKB Seq. No. P02788) comprising the amino acid sequence set forth in SEQ ID NO: 2 is particularly preferred.

The wild type transferrin protein contains two homologous lobes (N- and C-lobes) with each lobe binding a single iron atom. As such, each wild type transferrin molecule can bind up to two iron atoms or ions per molecule. Similarly, each wild type lactoferrin molecule can bind two iron atoms per molecule in an analogous fashion.

Transferrin and lactoferrin can be extracted from natural sources or alternatively manufactured using a recombinant production/manufacturing process. Suitable natural sources may be human plasma or human milk respectively.

By “transferrin” the current specification is to be construed as meaning a therapeutically effective amount of:
a wild type (mammalian, preferably human) transferrin protein,
a functional mutant thereof,
a functional fragment thereof, or
combinations thereof.

The iron saturation of the transferrin, functional mutant thereof, or functional fragment thereof may be about 50% or less. Preferably, the iron saturation is about 40% or less. In one embodiment, the iron saturation is about 30% or less. For example, the iron saturation may be about 20% or less, such as about 10% or less. In some embodiments, the iron saturation is about 5% or less. In yet a further embodiment, the iron saturation may be less than about 1%. For the avoidance of any doubt, ranges presented herein as less than X % include 0 to X %, i.e. transferrin with absolutely no bound iron—0% iron saturation.

As used herein, “apo-transferrin” shall mean transferrin having an iron saturation of less than 1%. Similarly, “holo-transferrin” shall mean transferrin having an iron saturation of 99% or greater.

The skilled person will appreciate that transferrin iron saturation levels can be readily determined without undue burden by quantifying the total iron levels in a sample having a known transferrin concentration. The total iron levels in sample can be measured by any one of a number of methods know by those of skill in the art. Suitable examples include:
Colorimetric assays—Iron is quantitated by measuring the intensity of the violet complex formed in the reaction between ferrozine and Fe²⁺ in acetate buffer at 562 nm. Thiourea or other chemicals can be added to complex contaminant metals such Cu²⁺, which can also bind with ferrozine and yield falsely elevated iron values. See Ceriotti et al., Improved direct specific determination of serum iron and total iron-binding capacity Clin Chem. 1980, 26(2), 327-31, the contents of which are incorporated herein by reference.
Inductively Coupled Plasma Atomic Emission Spectroscopy (ICP-AES)—is an emission spectroscopy technique that quantifies the mass percentage of metals in a sample. ICP-AES is based on the excitation of metal atoms/ions in the sample using a plasma (an ionized gas consisting of positive ions and free electrons) and analyzing the emission wavelength of the electromagnetic radiation, which is typical of that particular metal. Even though the technique is a standard analytical technique within common general knowledge of the skilled person, further information on ICP-AES can be found in Manley et al., Simultaneous Cu-, Fe-, and Zn-specific detection of metalloproteins contained in rabbit plasma by size-exclusion chromatography-inductively coupled plasma atomic emission spectroscopy. J Biol Inorg Chem. 2009, 14, 61-74, the contents of which are incorporated herein by reference.

The preferred method of determining the iron content of a sample for the purposes of the therapeutic method of the present invention is ICP-AES. The iron saturation of transferrin is then calculated based on the transferrin protein concentration, total iron content of the sample, and the fact that wild type transferrin has two iron-binding sites. Wild type human transferrin (molecular weight 79,750) can bind two iron atoms such that a sample containing 1 g of transferrin would be 100% saturated by 1.4 mg of iron.

Where the transferrin concentration of a particular sample is unknown it can be readily determined by a variety of well-characterized immunological (ELISA, nephelometry) and non-immunological methods (absorbance, AU480 chemical analysis).

At the time of writing, transferrin has not been authorized as a pharmaceutical in any major jurisdiction worldwide. As such, pharmacopoeial monographs do not exist for transferrin. Further information on the physical properties of transferrin, such as iron saturation can be obtained from the main reference text consulted by the skilled person; See L von Bonsdorff et al., Transferrin, Ch 21, pg 301-310, Production of Plasma Proteins for Therapeutic Use, Eds. J. Bertolini et al., Wiley, 2013 [Print ISBN:9780470924310 |Online ISBN:9781118356807], the contents of which are incorporated herein by reference and would be deemed to be within the common general knowledge of the skilled person.

By “lactoferrin” the current specification is to be construed as meaning a therapeutically effective amount of:
a wild type (mammalian, preferably human) lactoferrin protein,
a functional mutant thereof,
a functional fragment thereof, or
combinations thereof.

The iron saturation of the lactoferrin, functional mutant thereof, or functional fragment thereof may be about 50% or less. Preferably, the iron saturation is about 40% or less. In one embodiment, the iron saturation is about 30% or less. For example, the iron saturation may be about 20% or less, such as about 10% or less. In some embodiments, the iron saturation is about 5% or less. In yet a further embodiment, the iron saturation may be less than about 1%.

As used herein, “apo-lactoferrin” shall mean lactoferrin having an iron saturation of less than 1%. Similarly, “holo-lactoferrin” shall mean lactoferrin having an iron saturation of 99% or greater. Iron content, and saturation levels for lactoferrin can be measured analogously to those of transferrin discussed in detail above.

In using the terms transferrin and lactoferrin the present specification includes within its scope recombinant derivatives of transferrin and lactoferrin that differ from the wild type amino acid sequences of the human proteins, outlined in SEQ ID NOS: 1 & 2 respectively, by one or more substitutions, one or more deletions, or one or more insertions that may not materially alter the structure, or hydropathic nature of the recombinant proteins relative to the wild type proteins. Recombinant variants of transferrin and lactoferrin within the scope of the present invention may additionally comprise at least one post translational modification, such as pegylation, glycosylation, polysialylation, or combinations thereof.

In one embodiment, the present invention contemplates recombinant variants of transferrin and lactoferrin having one or more conservative substitutions relative to the wild type proteins in SEQ ID NOS: 1 & 2. A “conservative substitution” is one in which an amino acid is substituted for another amino acid that has similar properties, such that one skilled in the art of peptide chemistry would expect the secondary structure and hydropathic nature of the polypeptide to be substantially unchanged. Generally, change within the following groups of amino acids represent conservative changes: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his.

For example, the recombinant transferrin or lactoferrin within the scope of the method of treatment of the present invention may possess at least 90%, 95%, 96%, 97%, 98% or 99% homology with the wild type human transferrin and human lactoferrin proteins outlined in SEQ ID NO: 1 and SEQ ID NO: 2 respectively.

In a further embodiment, the present invention includes specific mutant forms of transferrin and/or lactoferrin that maintain their structure but prevent the protein binding iron at one or the other of the iron-biding domains, e.g. the N-lobe, the C-lobe, or a combination thereof.

Transferrin mutants within the scope of the present invention include, but are not limited to:
i) Y188F mutant N lobe (SEQ ID NO: 3);
ii) Y95F/Y188F mutant N lobe (SEQ ID NO: 4); and
iii) Y426F/Y517F mutant C lobe (SEQ ID NO: 5).

The skilled person will appreciate that recombinant proteins can be obtained utilising standard techniques well known in the art of protein expression, production and purification. Nucleic acid sequences of recombinant protein of interest can be inserted in any expression vector suitable for expression in the elected host cell, e.g. mammalian cells, insect cells, plant cells, yeast, and bacteria.

As used herein, the term “expression vector” refers to an entity capable of introducing a protein expression construct into a host cell. Some expression vectors also replicate inside host cells, which increases protein expression by the protein expression construct. One type of vector is a “plasmid,” which refers to a circular double stranded DNA loop into which additional DNA segments may be ligated. Other vectors include cosmids, bacterial artificial chromosomes (BAC) and yeast artificial chromosomes (YAC), fosmids, phage and phagemids. Another type of vector is a viral vector, wherein additional DNA segments may be ligated into the viral genome. Certain vectors are capable of autonomous replication in a host cell into which they are introduced (e.g., vectors having an origin of replication which functions in the host cell). Other vectors can be integrated into the genome of a host cell upon introduction into the host cell and are thereby replicated along with the host genome. Moreover, certain preferred vectors are capable of directing the expression of genes to which they are operatively linked.

Suitable bacterial cells include Escherichia coli, Bacillus subtilis, Salmonella typhimurium, Pseudomonas spp., Streptomyces spp., and Staphylococcus spp. Suitable yeast cells include Saccharomyces spp., Pichia spp., and Kuyveromyces spp. Suitable insect cells include those derived from Bombyx mori, Mamestra brassicae, Spodoptera frugiperda, Trichoplusia ni and Drosophila melanogaster. Such mammalian host cells include but are not limited to CHO, VERO, BHK, Hela, COS, MDCK, W138, BT483, Hs578T, HTB2, BT2O and 147D, NSO, CRL7O3O, HsS78Bst, human hepatocellular carcinoma cells (e.g. Hep G2), human adenovirus-transformed 293 cells (e.g. HEK293), PER.C6, mouse L-929 cells, HaK hamster cell lines, murine 313 cells derived from Swiss, Balb-c or NIH mice, and CV-1 cell line cells.

The present invention also contemplates the use of wild type and recombinant transferrin and lactoferrin proteins that are conjugated or fused to any other protein, protein fragment, protein domain, peptide, small molecule or other chemical entity. For example, suitable fusion or conjugation partners include serum albumins (for example, bovine, rabbit or human), keyhole limpet hemocyanin, immunoglobulin molecules (including the Fc domain of immunoglobulins), thyroglobulin, ovalbumin, tetanus toxoid, or a toxoid from other pathogenic bacteria, or an attenuated toxin derivative, cytokines, chemokines, glucagon-like peptide-1, exendin-4, XTEN, or combinations thereof.

In one embodiment of the invention, the transferrin and lactoferrin proteins used in the method of the present invention are fusion proteins having an improved in-vivo half-life, in which:
the wild type (mammalian, preferably human) transferrin or lactoferrin protein is fused to a fusion partner selected from an immunoglobulin Fc domain and albumin; or
a mutant transferrin or lactoferrin protein within the scope of the method of the present invention is fused to a fusion partner selected from an immunoglobulin Fc domain and albumin.

In one embodiment, the preferred fusion partner is an immunoglobulin Fc domain. For example, the immunoglobulin Fc domain may comprise at least a portion of a constant heavy immunoglobulin domain. The constant heavy immunoglobulin domain is preferably an Fc fragment comprising the CH2 and CH3 domain and, optionally, at least a part of the hinge region. The immunoglobulin Fc domain may be an IgG, IgM, IgD, IgA or IgE immunoglobulin Fc domain, or a modified immunoglobulin Fc domain derived therefrom. Preferably, the immunoglobulin Fc domain comprises at least a portion of a constant IgG immunoglobulin Fc domain. The IgG immunoglobulin Fc domain may be selected from IgG1, IgG2, IgG3 or IgG4 Fc domains, or modified Fc domains thereof.

In one embodiment, the fusion protein may comprise transferrin fused to an IgG1 Fc domain. In one embodiment, the fusion protein may comprise a transferrin mutant fused to an IgG1 Fc domain.

### Neurodegenerative Event

Surprisingly, the present inventors have discovered that transferrin and lactoferrin have an unexpected therapeutic role outside of iron binding/delivery to cells, in that both proteins were highly effective in stimulating the development of neural cells from neural progenitor cells and/or neural stem cells. The present invention thus provides for a method of stimulating neural cell development in a patient that has suffered a neurodegenerative event arising from at least one of a traumatic brain injury, a non-traumatic brain injury, a spinal cord injury, a peripheral nerve injury, or peripheral neuropathy,

the method comprising administering a therapeutically effective amount of a protein selected from transferrin, lactoferrin, and combinations thereof to the patient in need thereof.

As used herein, the term “stimulating neural cell development” is utilised to mean that the transferrin or lactoferrin are having a direct or indirect effect on neural progenitor cells and/or neural stem cells in the patient so as to produce new neural cells. Without wishing to limit the generality of the invention, it is postulated that the administration of transferrin or lactoferrin results in an increase in at least one of:
i) proliferation of the neural progenitor cells and/or neural stem cells within the patient, or
ii) inducing differentiation of the neural progenitor cells and/or neural stem cells into differentiated neural cells.
compared to neural progenitor cells/neural stem cells that have not been exposed to the transferrin, or lactoferrin.

By “neural cells”, the present specification includes all cells of the nervous system including without limitation glial cells, and neuronal cells. In one embodiment, the neural cells referred to in the method of the present invention are neuronal cells and the transferrin and lactoferrin potentiate the neurogenesis of new neuronal cells.

As used herein, the term “neurodegenerative event” refers to an event that causes the loss of structure and/or function of neural cells and includes the death of neural cells. The event may be an isolated one-off event/occurrence causing immediate neural cell damage or death. Alternatively, the event may be a continuous or chronic event that progressively leads to increasing levels of neural cell damage or death. In a particular embodiment, the neurodegenerative event causes the loss of structure, loss of function, or death of neuronal cells (or neurons) in the brain and/or spinal cord resulting in brain and/or spinal cord damage and dysfunction.

With respect to the method of the present invention, the neurodegenerative event is one arising from the consequences of at least one of a traumatic brain injury, a non-traumatic brain injury, a spinal cord injury, a peripheral nerve injury, or peripheral neuropathy.

As used herein, the term “traumatic brain injury” refers to an injury to the brain caused by a penetrating or non-penetrating trauma to the head. There are many possible causes, and non-limiting examples include road traffic accidents, assaults, sporting collisions, unprotected falls and the like.

As used herein, the term “non-traumatic brain injury” refers to an injury to the brain resulting from a non-traumatic cause. Suitable, non-limiting examples of non-traumatic causes include tumours, strokes, transient ischaemic attacks, brain haemorrhages, haemorrhagic strokes, toxins/drugs, cerebral hypoxia, cerebral anoxia, consumption of chemical toxins, hydrocephalus, meningitis and encephalitis. In one embodiment, the non-traumatic brain injury is caused by a stroke, such as an ischemic stroke or a haemorrhagic stroke. For example, the non-traumatic brain injury may be caused by an ischemic stroke.

Similarly, by “spinal cord injury” the present specification is to be construed as meaning damage to any part of the spinal cord or nerves at the end of the spinal canal that result in a loss of function, such as mobility and/or feeling. Non-limiting causes of spinal cord injury include trauma (car accident, gunshot, falls, etc.), disease (polio, spina bifida, etc.), infection and tumours.

In one embodiment, the patient may have suffered a neurodegenerative event arising from at least one of a stroke, a peripheral nerve injury, a traumatic brain injury, or peripheral neuropathy. For example, the patient may have suffered a neurodegenerative event arising from a peripheral nerve injury. In one embodiment, the patient may have suffered a neurodegenerative event arising from a stroke, such as an ischemic stroke or a haemorrhagic stroke. In one embodiment, the patient may have suffered a neurodegenerative event arising from an ischemic stroke.

As a non-limiting/non-binding theory, it is known that neurodegenerative insult or injury causes neural stem cells to migrate to the site of such insult or injury. See Arvidsson et al., 2002, Nat. Med., 8, 963-970; Kokaia and Lindvall, 2003, Curr. Opin. Neurobiol., 13, 127-132; and Kernie et al., 2010, Neurobiol. Disease, 37, 267-274. The present inventors postulate that by increasing the concentration of transferrin, lactoferrin, or combinations thereof within the patient such molecules can potentiate and/or promote the body's own neuroregenerative repair mechanisms. Transferrin and lactoferrin can be administered directly or indirectly to the site of neurodegenerative insult or injury by any conventional drug delivery means known by those skilled in the art.

It should be appreciated by those skilled in the art that the specific embodiments disclosed within above paragraphs should not be read in isolation, and that the present specification intends for these embodiments to be disclosed in combination with other embodiments as opposed to being disclosed individually. For example, each of the embodiments disclosed in above paragraphs is to be read as being explicitly combined with each of the embodiments in above paragraphs, or any permutation of 2 or more of the embodiments disclosed therein.

### Combination Therapy

The method of the present invention also contemplates the use of supplementary active compounds and molecules in combination with transferrin and/or lactoferrin. The supplementary active compounds and molecules can be co-formulated with transferrin, or lactoferrin as a unit dosage form, i.e. as a physically discrete unit intended as a unitary dosage for the subject to be treated. Alternatively, the supplementary active compounds and molecules can be presented as a kit-of-parts, and:
administered separately to transferrin and/or lactoferrin, in a phased or sequential dosing pattern; or
co-administered simultaneously from different dosage forms.

For example, the method of the present invention contemplates administering other serum or plasma-based proteins in combination with transferrin, and/or lactoferrin. Serum or plasma proteins within the scope of the present invention include those purified from a suitable plasma source, such as human plasma, and those prepared using recombinant manufacturing techniques. For example, the serum or plasma protein may be selected from the group consisting of Albumin (e.g. ALBUTEIN), Alpha-1 Antitrypsin/Alpha-1 Proteinase Inhibitor (e.g. PROLASTIN), Antithrombin (e.g. THROMBATE III), polyclonal immunoglobulins (IgG, IgA, and combinations thereof), polyspecific immunoglobulins (IgM), C1 esterase inhibitor (e.g. BERINERT), Transthyretin, and combinations thereof.

Exemplary polyclonal immunoglobulins within the scope of the present invention include commercially available polyclonal IgG formulations such as FLEBOGAMMA DIF 5% & 10%, GAMUNEX-C 10%, BIVIGAM 10%, GAMMAGARD Liquid 10%, etc.

Exemplary polyspecific immunoglobulins (IgM) within the scope of the present invention include commercially available immunoglobulin formulations containing polyspecific IgM such as PENTAGLOBIN or TRIMODULIN.

In one embodiment, the serum or plasma protein may be selected from the group consisting of Albumin, Antithrombin, Alpha-1 Antitrypsin, C1 esterase inhibitor, and combinations thereof. For example, the serum or plasma protein may be selected from the group consisting of Antithrombin, Alpha-1 Antitrypsin, and combinations thereof. In a particular embodiment, a therapeutically effective amount of Alpha-1 Antitrypsin is administered to the patient in addition to the protein selected from transferrin, lactoferrin, and combinations thereof. In a particular embodiment, a therapeutically effective amount of Antithrombin is administered to the patient in addition to the protein selected from transferrin, lactoferrin, and combinations thereof.

The method of the present invention also provides for administering known neurogenic/neurotrophic compounds and molecules in combination with transferrin and/or lactoferrin. For example, the method of the present invention contemplates administering neurogenic/neurotrophic proteins, peptides, and small molecules alongside transferrin and/or lactoferrin.

Suitable neurogenic and/or neurotrophic compounds and molecules may be selected from the group consisting of BDNF (brain-derived neurotrophic factor; NGF superfamily; SEQ ID NO: 6), GNDF (glial cell line-derived neurotrophic factor; TGF-β superfamily; SEQ ID NO: 7), CNTF (cilliary neurotrophic factor-1; neurokine superfamily; SEQ ID NO: 8), PACAP (amino acids 1-38 of pituitary adenylate cyclase-activating polypeptide; SEQ ID NO: 9), Y-27632 and pharmaceutically acceptable salts thereof [trans-4-[(1 R)-1-Aminoethyl]-N-4-pyridinylcyclohexanecarboxamide], Fasudil and pharmaceutically acceptable salts thereof [hexahydro-1-(5-isoquinolinyl-sulfonyl)-1H-1,4-diazepine], and combinations thereof.

The skilled person will appreciate that the present invention also contemplates within its scope covalent conjugates of each of the above listed compounds and molecules to each of transferrin and lactoferrin. Furthermore, it will be appreciated that the present invention also contemplates within its scope recombinant fusion proteins of each of the above listed proteins and peptides with each of transferrin and lactoferrin.

In one embodiment, the transferrin, lactoferrin, or combinations thereof may constitute at least 20% by weight of the total protein content utilised in the therapeutic method of the present invention. For example, the transferrin, lactoferrin, or combinations thereof may constitute greater than or equal to about 30%, 40%, 50%, 60%, 70%, 75%, 80%, 85%, 90%, 93%, 95%, 96%, 97%, 98%, or 99% by weight of the total protein content utilised in the combination therapy of the present invention.

It should be appreciated by those skilled in the art that the specific embodiments disclosed within above paragraphs should not be read in isolation, and that the present specification intends for these embodiments to be disclosed in combination with other embodiments as opposed to being disclosed individually. For example, each of the embodiments disclosed in above paragraphs is to be read as being explicitly combined with each of the embodiments in above paragraphs, or any permutation of 2 or more of the embodiments disclosed therein.

### Pharmaceutical Compositions of the Invention

In a further aspect, the present invention also provides for a pharmaceutical composition comprising transferrin, lactoferrin, or combinations thereof for use in the generation of new neural cells in a patient that has suffered a neurodegenerative event arising from a traumatic brain injury, a non-traumatic brain injury, a spinal cord injury, and combinations thereof.

The pharmaceutical compositions of the present invention may optionally further comprise at least one pharmaceutically acceptable carrier. The at least one pharmaceutically acceptable carrier may be chosen from adjuvants and vehicles. The at least one pharmaceutically acceptable carrier includes any and all solvents, diluents, other liquid vehicles, dispersion aids, suspension aids, surface active agents, isotonic agents, thickening agents, emulsifying agents, preservatives, as suited to the particular dosage form desired.

Suitable carriers are described in Remington: The Science and Practice of Pharmacy, 21 st edition, 2005, ed. D. B. Troy, Lippincott Williams & Wilkins, Philadelphia, and Encyclopedia of Pharmaceutical Technology, eds. J. Swarbrick and J. C. Boylan, 1988-1999, Marcel Dekker, New York, the contents of which are incorporated herein by reference. Preferred examples of such carriers or diluents include, but are not limited to, water, saline, Ringer's solutions, glycols, dextrose solution, buffered solutions (such as phosphates, glycine, sorbic acid, and potassium sorbate) and 5% human serum albumin. Liposomes and non-aqueous vehicles such as glyceride mixtures of saturated vegetable fatty acids, and fixed oils (such as peanut oil, cottonseed oil, safflower oil, sesame oil, olive oil, com oil and soybean oil) may also be used depending on the route of administration.

The pharmaceutical composition of the invention is formulated to be compatible with its intended route of administration. For systemic use, the pharmaceutical composition of the invention can be formulated for administration by a conventional route selected from the group consisting of intravenous, subcutaneous, intramuscular, intradermal, intraperitoneal, intracerebral, intracranial, intrapulmonary, intranasal, intraspinal, intrathecal, transdermal, transmucosal, oral, vaginal, and rectal.

In one embodiment, parenteral administration is the preferred route of administration. The pharmaceutical composition may be enclosed in ampoules, disposable syringes, sealed bags, or multiple dose vials made of glass or plastic. In a one embodiment, administration as an intravenous injection is the preferred route of administration. The formulations can be administered continuously by infusion or by bolus injection.

The pharmaceutical compositions of the present invention may be presented as a unit dosage unit form, i.e. as physically discrete units intended as unitary dosages for the subject to be treated.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, CREMOPHOR EL, or phosphate buffered saline (PBS). In all cases, the composition must be sterile and should be fluid to the extent that easy syringe-ability exists.

The compositions of the invention should be stable under the conditions of manufacture and storage. Moreover, compositions should be preserved against the contaminating action of microorganisms such as bacteria and fungi. The carrier can be a solvent or dispersion medium containing, for example water, ethanol, polyols (for example, glycerol, propylene glycol, and liquid polyethylene glycol, and the like), and suitable mixtures thereof.

Prevention of the action of microorganisms can be achieved by various antibacterial and antifungal agents, for example parabens, chlorobutanol, phenol, ascorbic acid, thimerosal, and the like. In many cases, it will be preferable to include isotonic agents, for example sugars (such as mannitol, sorbitol, etc.), polyalcohols, or sodium chloride in the composition. Prolonged absorption of the injectable compositions can be brought about by including in the composition an agent which delays absorption, for example aluminum monostearate or gelatin.

Sterile injectable solutions of the pharmaceutical composition of the present invention can be prepared by incorporating the active molecule in the required amount in an appropriate solvent with one or a combination of ingredients as discussed above followed by filtered sterilization. In the case of sterile powders for the preparation of sterile injectable solutions, methods of preparation include vacuum drying and freeze-drying that provide a powder of the active ingredient plus any additional desired ingredient from a previously sterile-filtered solution thereof.

Except insofar as any conventional media or agent is incompatible with the active molecules of the present invention use thereof in the compositions is contemplated to be within the scope of the present invention.

In one embodiment, the transferrin, lactoferrin, or combinations thereof may constitute at least 20% by weight of the total protein content of the pharmaceutical composition of the present invention. For example, the transferrin, lactoferrin, or combinations thereof may constitute greater than or equal to about 30%, 40%, 50%, 60%, 70%, 75%, 80%, 85%, 90%, 93%, 95%, 96%, 97%, 98%, or 99% by weight of the total protein content of the pharmaceutical composition of the present invention.

It should be appreciated by those skilled in the art that the specific embodiments disclosed within above paragraphs should not be read in isolation, and that the present specification intends for these embodiments to be disclosed in combination with other embodiments as opposed to being disclosed individually. For example, each of the embodiments disclosed in above paragraphs is to be read as being explicitly combined with each of the embodiments in above paragraphs, or any permutation of 2 or more of the embodiments disclosed therein.

### Dosing

As discussed supra, the present inventors postulate that by increasing the concentration of transferrin, lactoferrin, or combinations thereof proximate to the site of a neurodegenerative insult or injury, such molecules can potentiate and/or promote the body's own neuroregenerative repair mechanisms. Transferrin and lactoferrin could be administered directly or indirectly to the site of neurodegenerative insult or injury by any conventional drug delivery means known by those skilled in the art. For example, the transferrin, lactoferrin, or combinations thereof could be administered locally or proximate to the injury caused by the neurodegenerative event by a conventional route selected from the group consisting of intracerebral, intracranial, intraspinal, and intrathecal. For example, the transferrin, lactoferrin, and combinations thereof may be administered locally during surgical intervention.

Alternatively, the transferrin, lactoferrin, or combinations thereof could be delivered indirectly to the site of the neurodegenerative insult or injury by an administration route selected from the group consisting of intravenous, subcutaneous, intramuscular, intradermal, intraperitoneal, intrapulmonary, intranasal, transdermal, transmucosal, oral, vaginal and rectal.

For the avoidance of any doubt, the opportunity is taken to clarify that the present specification speaks to transferrin iron saturation levels in two separate and distinct contexts:
a) In the first context, as outlined, the specification is speaking to the iron saturation of purified exogenous transferrin in a pharmaceutical composition that is to be administered to a patient. In this instance, iron saturation levels of the purified exogenous transferrin can be determined using Inductively Coupled Plasma Atomic Emission Spectroscopy (but other methods, such as colorimetric methods can also be used).
b) In the second context, which will be discussed in more detail immediately below, the present specification is speaking to measuring the iron saturation of physiological transferrin in a patient, i.e. in the patient's plasma or serum, after the pharmaceutical composition containing exogenous transferrin has been administered to the patient.

Under normal physiological conditions, practically all iron in plasma is bound to transferrin and the resulting iron saturation of physiological transferrin is approximately 30%. In Example 6 (vide infra), the present inventors have demonstrated that transferrin with an iron saturation of less than 30% results in an unexpected neuroregenerative effect. As a non-limiting hypothesis, it is envisaged that by administering a pharmaceutical composition containing exogenous transferrin (with a low iron saturation) to a patient that the physiological concentrations of transferrin within the patient's plasma will increase resulting in the iron saturation of physiological transferrin dropping below 30%. Thus, allowing physiological transferrin to leverage a neuroregenerative effect. Naturally, exogenous transferrin with an iron saturation of less than 1% will likely be more efficacious than exogenous transferrin with an iron saturation 40%.

Accordingly, in one embodiment, the protein selected from transferrin, lactoferrin, and combinations thereof is administered to the patient at a concentration sufficient to reduce the iron saturation of the patient's transferrin (in a serum or plasma sample of the patient) below about 30%. Preferably, the protein selected from transferrin, lactoferrin, and combinations thereof is administered to the patient at a concentration sufficient to reduce the iron saturation of the patient's transferrin (in a serum or plasma sample of the patient) below about 20%, for example below about 10%. The transferrin, lactoferrin, or combinations thereof may be administered to the patient using a titration based-dosage regimen to achieve this level of serum or plasma transferrin iron saturation.

The skilled person will appreciate that the measurement of transferrin iron saturation levels in a patient's serum or plasma is a routine assay typically performed using colorimetric methodologies as discussed supra. Plasma or serum iron content, is measured on chemical analyzers by using a colorimetric reaction with ferene or ferrozine as a chromogen to form a colour complex with iron. An analysed sample produces two values:
sample iron content (i.e. iron bound to transferrin in the sample), and unsaturated iron binding capacity (UIBC, i.e. the number of unoccupied iron biding sites on transferrin in the sample).
Total iron binding capacity (TIBC) is the sum of the sample iron content and UIBC.
Transferrin saturation (%) is determined as [(sample iron content/TIBC)×100].

The workings of colorimetric assays for the measurement of transferrin iron saturation levels in a patient's serum or plasma are common general knowledge and further information can be found in various literature reviews, such as Pfeiffer et al., Am J Clin Nutr 2017, 106(Suppl), 1606S-14S, the contents of which are incorporated herein by reference.

In yet a further embodiment of the method of the present invention the protein selected from transferrin, lactoferrin, and combinations thereof can be administered to the patient at a concentration of from about 5 mg/kg to about 8400 mg/kg. For example, from about 10 mg/kg to about 7000 mg/kg, such as from about 20 mg/kg to about 6000 mg/kg, for example from about 50 mg/kg to about 5000 mg/kg. In some embodiments the protein selected from transferrin, lactoferrin, and combinations thereof can be administered to the patient at a concentration of from about 50 mg/kg to about 1000 mg/kg. Suitably, the protein can be administered at a concentration of from about 50 mg/kg to about 500 mg/kg, such as from about 50 mg/kg to about 250 mg/kg, for example from about 50 mg/kg to about 150 mg/kg.

In one embodiment, the method of the present invention may comprise administering the protein selected from transferrin, lactoferrin, and combinations thereof to a patient in need thereof as part of a multiple dosing regimen. For example, at initial dose of about 50 mg/kg to about 5000 mg/kg on day 1 of an administration period, followed by about 50 mg/kg to about 1000 mg/kg per dose during a multiple dosing period. For example, at initial dose of about 50 mg/kg to about 1000 mg/kg on day 1 of an administration period, followed by about 50 mg/kg to about 500 mg/kg per dose during a multiple dosing period. For example, at initial dose of about 50 mg/kg to about 500 mg/kg on day 1 of an administration period, followed by about 50 mg/kg to about 250 mg/kg per dose during a multiple dosing period. For example, at initial dose of about 50 mg/kg to about 250 mg/kg on day 1 of an administration period, followed by about 50 mg/kg to about 250 mg/kg per dose during a multiple dosing period. The multiple dosing period may comprise from about 3 to about 30 administrations up to a total cumulative dose. The multiple dosing period may be from about 1 to about 30 weeks. The multiple portion doses may be administered at intervals of from about 1 day to about 30 day.

It should be appreciated by those skilled in the art that the specific embodiments disclosed within above paragraphs should not be read in isolation, and that the present specification intends for these embodiments to be disclosed in combination with other embodiments as opposed to being disclosed individually. For example, each of the embodiments disclosed in above paragraphs is to be read as being explicitly combined with each of the embodiments in above paragraphs, or any permutation of 2 or more of the embodiments disclosed therein.

### BRIEF DESCRIPTION OF THE DRAWINGS

Additional features and advantages of the present invention will be made clearer in the appended drawings, in which:

FIGS. 1A to 1D show the induction of neurite outgrowth, and proliferation in SH-SY5Y cells and increased β-III-tubulin protein concentrations in response to apo-transferrin;

FIGS. 2A to 2B demonstrate that apo-transferrin induces primary human neural progenitor cells to become β-III-tubulin protein positive neurons and GFAP protein positive astrocytes cells;

FIG. 3A plots the effect of deferoxamine mesylate at various concentrations relative to apo-transferrin on neurite outgrowth in SH-SY5Y cells;

FIG. 3B illustrates the efficacy of a transferrin mutant having reduced iron binding capacity on promoting neurite outgrowth in SH-SY5Y cells;

FIG. 4 plots the effect of various different proteins on neurite outgrowth in SH-SY5Y cells;

FIG. 5 plots the effect of IOX2, a prolyl hydroxylase inhibitor, on neurite outgrowth in SH-SY5Y cells;

FIGS. 6A & 6B illustrate the role of iron saturation on the efficacy of transferrin in promoting neurite outgrowth in SH-SY5Y cells;

FIGS. 7A to 7D plots the effect of apo-transferrin in combination with other neurotrophic proteins/peptide fragments on neurite outgrowth in SH-SY5Y cells;

FIG. 8 plots the effect of apo-transferrin in combination with the small molecule Y-27632 on neurite outgrowth in SH-SY5Y cells;

FIGS. 9A and 9B illustrate that apo-transferrin increases the amount of neurogenesis as measured by new neuroblasts (as defined by BrdU+/DCX+ cells) and newly formed mature neurons (as defined by BrdU+/NeuN+ cells) in an animal test model; and

FIGS. 10A to 10C demonstrate that treatment of animals with apo-transferrin after transient MCAo results in quicker recovery, better motor skills, and higher cognition compared to saline-treated mice.

### DETALED EXAMPLES OF THE INVENTION

It should be readily apparent to one of ordinary skill in the art that the examples disclosed herein below represent generalised examples only, and that other arrangements and methods capable of reproducing the invention are possible and are embraced by the present invention.

### Example 1: Apo-Transferrin (ApoTf) Induces Differentiation and Neurite Outgrowth in SH-SY5Y Cells in a Dose Responsive Manner

Transferrin is utilized in cell culture and in-vivo to deliver iron as a nutrient to cells. This is typically accomplished through the actions of holo-transferrin (HoloTf) binding to, and endocytosis by, its cognate receptor CD71, the transferrin receptor 1 (TfR1). Transferrin is typically believed to provide cells with iron as a means to promote and sustain metabolic activity. The present inventors have surprisingly found that apo-transferrin, the iron-free form of transferrin protein, induces differentiation of a very common research model of neurons, SH-SY5Y cells. Induction of neuronal differentiation was assessed by morphological parameters of neurite formation (a key element typically used as a marker of neuronal differentiation, neuronal health, and function) according to the procedures of Agholme, 2010. J. of Alzheimer's Disease. Vol. 20:1p 069-108; and Dyberg et al., 2017. PNAS Vol 114 (32), E6603-E6612.

Undifferentiated SH-SY5Y cells were seeded into 96 well clear bottom plates in media containing 0.1% FBS. A serum-free base media was utilized as recommended by the supplier for SH-SY5Y cells (Sigma, Cat #94030304-1VL). Twenty-four hours after seeding cells, a 3× stock solution of ApoTf, final concentrations indicated on the x-axis, in serum free base media was added to the cells. ApoTf was obtained and purified from pooled human plasma and dosed at a final concentration of 0.2 mg/mL.

Cells were allowed to differentiate for 6 days. Neurite growth was assessed by imaging and image analysis. At the time of analysis, a 10× solution of Tubulin Tracker (Molecular Probes, T34075) and Hoechst 33342 (Molecular Probes #H3570) nuclear stain was prepared.

Briefly, Tubulin Tracker dissolved in DMSO was diluted 1:1 with Pluronic F-127 and further diluted into HBSS to generate a 10× solution. Hoechst 33342 was added to the HBSS-Tubulin Tracker solution at 10 μg/mL to generate a 10× nuclear stain. The 10× staining solution (10 μL) was added directly to treated assay wells and incubated at 37° C. for 30 minutes. Following incubation, 110 μL of 0.4% Trypan Blue was added directly to assay wells and imaged on a Molecular Devices Nano imaging instrument. Nine images/well were acquired in the blue (Nuclei) and green (Tubulin) fluorescent channels for each image.

After obtaining images, the MetaExpress Neurite Outgrowth analysis module (Molecular Devices) was used to identify cells, cell bodies, and quantify neurites. The total number of neurite branches were divided by the total number of cells imaged to account for different numbers of cells in each test well. The Outgrowth Fold Change was determined by setting the untreated control cells to a value of 1 with all other treatments shown relative to untreated control.

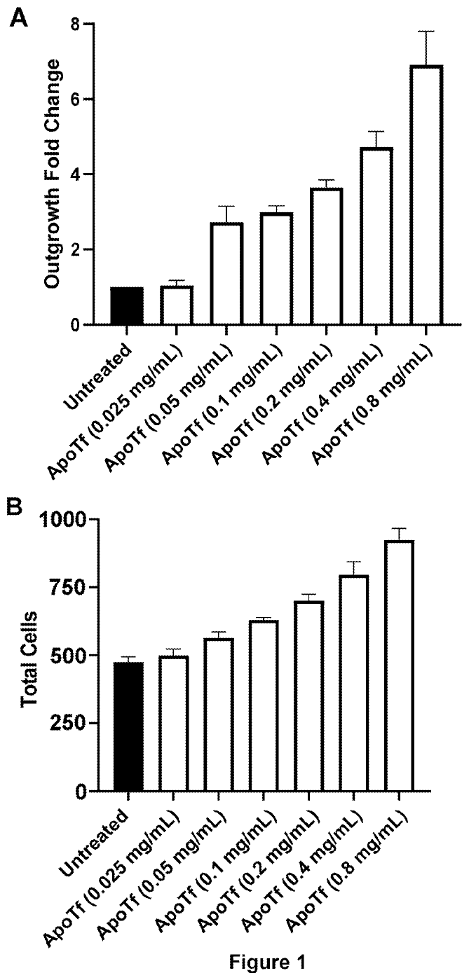

From FIG. 1A it is evident that apo-transferrin was able to induce neurite outgrowth in a dose dependent manner. Incremental increases in apo-transferrin concentration, up to a maximum of 0.8 mg/mL, were associated with an improved outgrowth response in the SH-SY5Y cells. This phenomenon is counterintuitive to the known function of transferrin, which primarily acts in the holo- or iron-laden form of transferrin.

FIG. 1B illustrates that apo-transferrin induces a concentration-dependent increase in cell numbers. Increased cell number is indicative of increased cell proliferation, up to the maximum tested dose of 0.8 mg/mL apo-transferrin.

FIG. 1C provides visual comparison of SH-SY5Y cells treated with 0.1 mg/mL ApoTf (lower panels) to an untreated control (upper panels). Left panels show nuclear staining with Hoechst 33342. Right images show tubulin staining of cell bodies and neurites. From FIG. 1C it is apparent that ApoTf had a profound effect on promoting cell proliferation, and subsequently/simultaneously promoting induction of neurite/tubulin outgrowth.

Additionally, as shown in FIG. 1D, it was found that apo-transferrin treatment caused an increase in β-III-tubulin protein, a well-characterized, traditional marker of neurons. In this experiment, the SH-SY5Y cells were differentiated as described supra. At the time of analysis, cells were fixed with paraformaldehyde, stained for β-III-tubulin (R&D Systems, MAB1195), and imaged on a Molecular Devices Nano imaging instrument. Image analysis was performed by assessing the fluorescent intensity of cells stained with β-III-tubulin. Background from secondary antibody alone was subtracted from all values. Values are shown with standard deviations as “β-III-Tubulin Staining Intensity” for the indicated conditions.

### SH-SY5Y Cells

By “SH-SY5Y cells” the present specification means a subcloned cell line derived from the SK-N-SH neuroblastoma cell line. It serves as a model for neurodegenerative disorders since the cells can be converted to various types of functional neural cells by the addition of specific compounds. In addition, the SH-SY5Y cell line has been used widely in experimental neurological studies, including analysis of neuronal differentiation, metabolism, and function related to neurodegenerative processes, neurotoxicity, and neuroprotection.

Outlined herein under are peer reviewed citations referencing the SH-SY5Y cell line as a predictive model for various neurodegenerative disorders. The list does not constitute an admission of prior art by the inventors, rather it serves to illustrate the skilled person's knowledge of the SH-SY5Y cell line as a predictive model for brain injuries and neuropathies.

### Neurogenesis

Dayem et al. Biologically synthesized silver nanoparticles induce neuronal differentiation of SH-SY5Y cells via modulation of reactive oxygen species, phosphatases, and kinase signaling pathways. Biotechnol. J. 2014, 9, 934-943.

Fagerstrom et al. Protein Kinase C-epsilon Implicated in Neurite Outgrowth in Differentiating Human Neuroblastoma Cells. Cell Growth & Differentiation Vol. 7, 775-785, June 1996.

### Peripheral Nerve Injury

Han et al. Berberine. Promotes Axonal Regeneration in Injured Nerves of the Peripheral Nervous System. J Med Food 15 (4) 2012, 413-417.

Gold et al. Nonimmunosuppressant FKBP-12 Ligand Increases Nerve Regeneration. EXPERIMENTAL NEUROLOGY 147, 269-278 (1997).

Kim et al. Protective effect of GCSB-5, an herbal preparation, against peripheral nerve injury in rats. Journal of Ethnopharmacology 136 (2011) 297-304.

Lesma et al. Glycosaminoglycans in Nerve Injury: I. Low Doses of Glycosaminoglycans Promote Neurite Formation. Journal of Neuroscience Research. 1996 46(5):565-71.

### Diabetic Neuropathy

Hattangady and Rajadhyaksha. A brief review of in vitro models of diabetic neuropathy. Int J Diabetes Dev Ctries. 2009 October-December; 29(4): 143-149.

Vincent et al. Oxidative Stress and Programmed Cell Death in Diabetic Neuropathy. Ann. N.Y. Acad. Sci. 959: 368-383 (2002).

Shindo. Modulation of Basal Nitric Oxide-dependent Cyclic-GMP Production by Ambient Glucose, Myo-Inositol, and Protein Kinase C in SH-SY5Y Human Neuroblastoma Cells. J. Clin. Invest. Volume 97, Number 3, February 1996, 736-745.

Li et al. C-peptide enhances insulin-mediated cell growth and protection against high glucose-induced apoptosis in SH-SY5Y cells. Diabetes Metab Res Rev 2003; 19: 375-385.

### Cancer Drug Induced Neuropathy

Rigolio et al. Resveratrol interference with the cell cycle protects human neuroblastoma SH-SY5Y cell from paclitaxel-induced apoptosis. Neurochemistry International 46 (2005) 205-211.

Donzelli et al. Neurotoxicity of platinum compounds: comparison of the effects of cisplatin and oxaliplatin on the human neuroblastoma cell line SH-SY5Y. Journal of Neuro-Oncology 67: 65-73, 2004.

Mannelli et al. Oxaliplatin-induced oxidativestressinnervoussystem-derived cellular models:Could it correlate with in vivo neuropathy? Free Radical Biology and Medicine 61 (2013) 143-150.

### Organophosphate Induced Neuropathy (Insecticides, Chemical Warfare Compounds)

Hong et al. Neurotoxicity induced in differentiated SK-N-SH-SY5Y human neuroblastoma cells by organophosphorus compounds. Toxicology and Applied Pharmacology 186 (2003) 110-118.

Ehrich et al. Interaction of organophosphorus compounds with muscarinic receptors in SH-SY5Y human neuroblastoma cells. Journal of Toxicology and Environmental Health 1994 43(1):51-63.

### Traumatic Brain Injury

Triyoso and Good. Pulsatile shear stress leads to DNA fragmentation in human SH-SY5Y neuroblastoma cell line. Journal of Physiology (1999), 515.2, pp. 355-365.

Song et al. Arctigenin Confers Neuroprotection Against Mechanical Trauma Injury in Human Neuroblastoma SH-SY5Y Cells by Regulating miRNA-16 and miRNA-199a Expression to Alleviate Inflammation. J Mol Neurosci (2016) 60:115-129.

Skotak et al. An in vitro injury model for SH-SY5Y neuroblastoma cells: Effect of strain and strain rate. Journal of Neuroscience Methods 205 (2012) 159-168.

Arun et al. Studies on blast traumatic brain injury using in-vitro model with shock tube. NeuroReport (2011) 22:379-384.

### Ischemia

Miglio et al. Cabergoline protects SH-SY5Y neuronal cells in an in vitro model of ischemia. European Journal of Pharmacology 489 (2004) 157-165.

Duong et al. Multiple protective activities of neuroglobin in cultured neuronal cells exposed to hypoxia re-oxygenation injury. J. Neurochem. (2009) 108, 1143-1154.

Qiu et al. Enhancement of ischemia-induced tyrosine phosphorylation of Kv1.2 by vascular endothelial growth factor via activation of phosphatidylinositol 3-kinase. J. Neurochem. (2003) 10.104.

### Example 2: The Effect of ApoTf on β-III-Tubulin and GFAP Protein Concentrations in Primary Human Neural Progenitor Cells

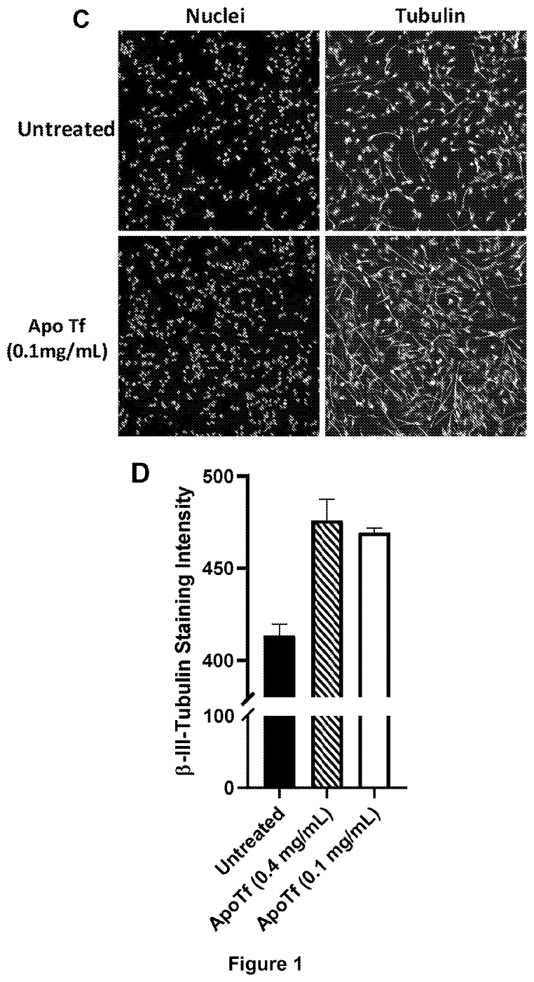

The neurogenic effects of ApoTf also translate to primary human brain cortex-derived neural progenitor cells, another established model of adult neurogenesis (See Azari and Reynolds, “In Vitro Models for Neurogenesis”. Cold Spring Harb Perspect Biol 2016, 8, a021279). As shown in FIGS. 2A and 2B, apo-transferrin dramatically increases the percentage of cells differentiated to neurons (% β-III-tubulin positive cells, 2A) and astrocytes (% GFAP positive cells, 2B), relative to cells without apo-transferrin, from a culture of primary human brain-derived neural progenitor cells.

Neural progenitor cells maintained as neurospheres were obtained from Lonza (PT-2599). Cells were thawed from a frozen vial of neurospheres and cultured in Human NeuroCult™ NS-A Complete Proliferation media (Stemcell Technologies) for 2 weeks. Neurospheres were dissociated to single cells and plated in Laminin coated wells of assay plates. The neural progenitor cells were seeded in NeuroCult™ NS-A Basal media containing 1/10th concentration of the recommended proliferation supplements, in the absence or presence of ApoTf (0.8 mg/mL) for 72 hours. At the time of analysis, cells were fixed with paraformaldehyde, stained for β-III-tubulin (R&D Systems, MAB1195) and GFAP (Invitrogen, PA3-16727), and imaged on a Molecular Devices Nano imaging instrument. Image analysis was performed by assessing the relative numbers of cells staining positive for β-III-tubulin or GFAP. Values for the indicated conditions are shown with standard deviations as “% β-III-Tubulin Positive” cells (FIG. 2A) or “% GFAP Positive” cells (FIG. 2B).

### Example 3: Iron Chelation is Not the Sole Mode of Action for Neurogenesis by ApoTf

Deferoxamine mesylate (DFO) is a small molecule iron chelator utilized in clinical practice for iron overload. Like ApoTf, DFO has high affinity binding constants for iron; although only a single iron binding site. The effect of DFO on neurite outgrowth was investigated. ApoTf was tested at a concentration near the bottom of its functional dose curve and compared to DFO's ability to induce neurite outgrowth. ApoTf tested at 2.4 μM (0.2 mg/mL) has two iron-binding sites and therefore is comparable to the single iron binding site of DFO at 4.8 μM.

Undifferentiated SH-SY5Y cells were seeded and treated as described in Example 1. Neurite growth was assessed by imaging and image analysis. At the time of analysis, a 10× solution of Tubulin Tracker (Molecular Probes, T34075) and Hoechst 33342 (Molecular Probes #H3570) nuclear stain was prepared. Briefly, Tubulin Tracker dissolved in DMSO was diluted 1:1 with Pluronic F-127 and further diluted into HBSS to generate a 10× solution. Hoechst 33342 was added to the HBSS-Tubulin Tracker solution at 10 μg/mL to generate at 10× nuclear stain. The 10× staining solution (10 μL) was added directly to treated assay wells and incubated at 37° C. for 30 minutes. Following incubation, 110 μL of 0.4% Trypan Blue was added directly to assay wells and imaged on a Molecular Devices Nano imaging instrument. Nine images/well were acquired in the blue (Nuclei) and green (Tubulin) fluorescent channels for each image. After obtaining images, the MetaExpress Neurite Outgrowth analysis module (Molecular Devices) was used to identify cell bodies and quantify neurites. The total number of neurite branches were divided by the total number of cells imaged to account for different numbers of cells. The Outgrowth Fold Change was determined by setting the untreated control to a value of 1 with all other treatments shown relative to untreated control. ApoTf was obtained and purified from pooled human plasma and dosed at a final concentration of 0.2 mg/mL. Deferoxamine mesylate (DFO) was obtained from Tocris (Cat #5764), resuspended and stored by the manufacturer's recommendations. Concentrations of DFO that were assessed for neurogenic properties are indicated on the x-axis.

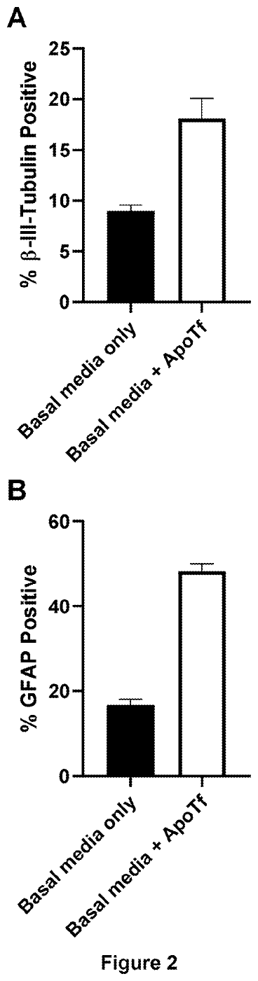

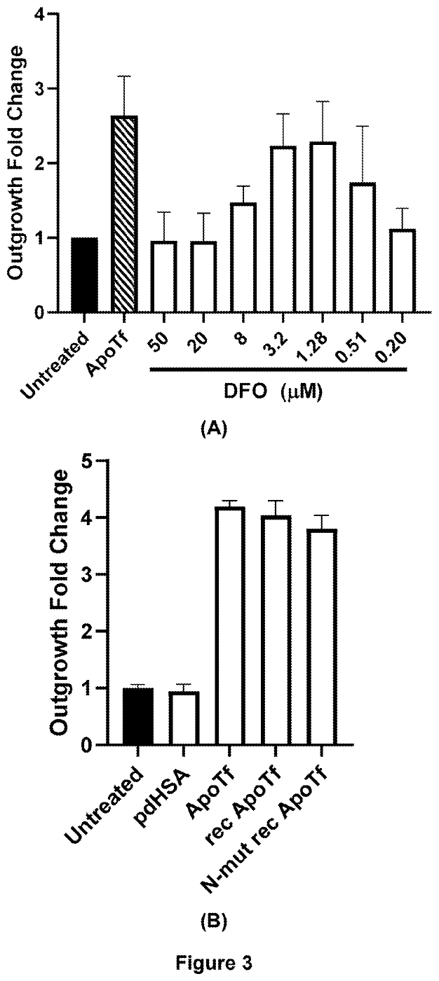

From FIG. 3 it can been seen that DFO shows maximal neurite outgrowth between 1-3 μM, with little neurite formation beyond that concentration, whereas ApoTf continues to increase differentiation even up to 9.9 μM (0.8 mg/mL; 20 μM iron binding sites). These data suggest that while iron chelation may play a role in neurite outgrowth, it is not the primary mechanism-of-action; another unidentified functional aspect of ApoTf must also play a role in its neurogenic ability.

The present inventors further sought to determine if a reduction of transferrin's iron-binding activity by mutation of the N-terminal iron-binding site was sufficient to mediate neurogenesis. Undifferentiated SH-SY5Y cells were treated as described in Example 1. Neurite growth was assessed by imaging and image analysis. At the time of analysis, a 10× solution of Tubulin Tracker (Molecular Probes, T34075) and Hoechst 33342 (Molecular Probes #H3570) nuclear stain was prepared. Briefly, Tubulin Tracker dissolved in DMSO was diluted 1:1 with Pluronic F-127 and further diluted into HBSS to generate a 10× solution. Hoechst 33342 was added to the HBSS-Tubulin T racker solution at 10 μg/mL to generate at 10× nuclear stain. The 10× staining solution (10 μL) was added directly to treated assay wells and incubated at 37° C. for 30 minutes. Following incubation, 110 μL of 0.4% Trypan Blue was added directly to assay wells and imaged on a Molecular Devices Nano imaging instrument. Nine images/well were acquired in the blue (Nuclei) and green (Tubulin) fluorescent channels for each image. After obtaining images, the MetaExpress Neurite Outgrowth analysis module (Molecular Devices) was used to identify cell bodies and quantify neurites.

The total number of neurite branches were divided by the total number of cells imaged to account for different numbers of cells. The Outgrowth Fold Change was determined by setting the untreated control to a value of 1 with all other treatments shown relative to untreated control. All proteins were dosed at a final concentration of 0.2 mg/mL.

Plasma-derived human serum albumin (pdHSA) and ApoTf were obtained and purified from pooled human plasma; recombinant ApoTf (rec ApoTf; SEQ ID NO: 1), and the N-lobe mutant Tf (N-mut rec ApoTf; SEQ ID 4) were obtained by cell culture expression from 293-6E cells.

Briefly, wild-type human transferrin (SEQ ID NO:1) and N-lobe mutant human transferrin (SEQ ID 4) sequences were cloned into mammalian expression plasmids containing N-terminal 6xHIS tag and TEV cleavage sites. The expression plasmids were transfected into the 293-6E cell line, with subsequent harvest of proteins from the cell culture supernatant. Proteins were purified on NI-NTA columns and eluted after washing. TurboTEV protease was used to cleave the N-terminal 6xHIS tag and additional amino acids from the transferrin proteins. Following TEV cleavage, the transferrin proteins were separated from cleaved 6xHIS tag and uncleaved protein by a second Ni-NTA capture column. The flow-through fraction of Ni-NTA capture column was then subject to low pH treatment to remove any potential residual iron bound to these proteins, buffer exchanged to PBS pH 7.4, concentrated, and sterile filtered for final use.

From FIG. 3B we see that plasma-derived human serum albumin (pdHSA) did not affect neurogenesis. However, both ApoTf and recombinant ApoTf did induce neurogenesis of SH-SY5Y. The ApoTf mutant (N-mut rec ApoTf) with reduced iron-binding capacity was almost equal to that of ApoTf and rec ApoTf at inducing differentiation of the SH-SY5Y cells. Iron-binding does not appear to be the sole mechanism of action for the neurogenic potential of ApoTf.

### Example 4: Neurogenic Effects on SH-SY5Y are Specific to Apo-Transferrin and Apo-Lactoferrin

As the role of iron chelation in ApoTf's neurogenic ability was found to be unclear from Example 3 the present inventors determined whether other iron binding proteins can also mediate neurogenesis of SH-SY5Y cells.

Undifferentiated SH-SY5Y cells were treated as described in Example 1. Neurite growth was assessed by imaging and image analysis. At the time of analysis, a 10× solution of Tubulin Tracker (Molecular Probes, T34075) and Hoechst 33342 (Molecular Probes #H3570) nuclear stain was prepared. Briefly, Tubulin Tracker dissolved in DMSO was diluted 1:1 with Pluronic F-127 and further diluted into HBSS to generate a 10× solution. Hoechst 33342 was added to the HBSS-Tubulin Tracker solution at 10 μg/mL to generate at 10× nuclear stain. The 10× staining solution (10 μL) was added directly to treated assay wells and incubated at 37° C. for 30 minutes. Following incubation, 110 μL of 0.4% Trypan Blue was added directly to assay wells and imaged on a Molecular Devices Nano imaging instrument. Nine images/well were acquired in the blue (Nuclei) and green (Tubulin) fluorescent channels for each image. After obtaining images, the MetaExpress Neurite Outgrowth analysis module (Molecular Devices) was used to identify cell bodies and quantify neurites. The total number of neurite branches were divided by the total number of cells imaged to account for different numbers of cells. The Outgrowth Fold Change was determined by setting the untreated control to a value of 1 with all other treatments shown relative to untreated control. BSA was obtained from Sigma; rHSA was obtained from Albumedix; ApoTf and HoloTf were obtained and purified from pooled human plasma; Apo-ferritin (equine) was obtained from Sigma; apo-lactoferrin was obtained from Athens Research & Technology. All proteins were dosed at a final concentration of 0.2 mg/mL.

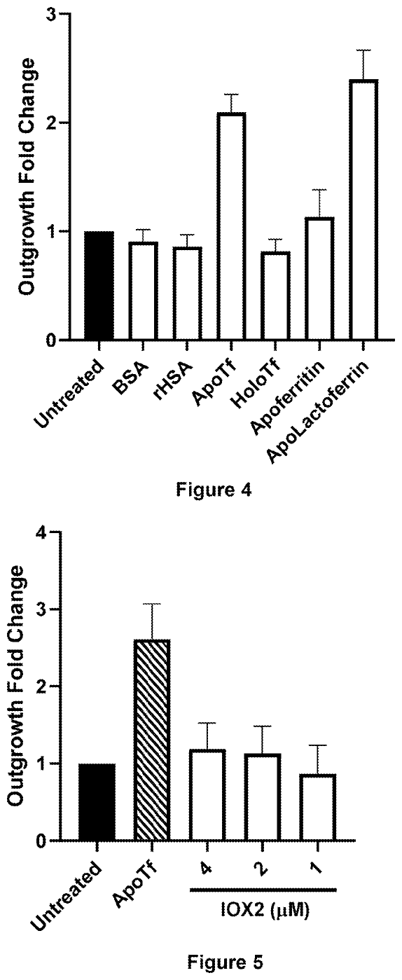

From FIG. 4 we see that neither bovine serum albumin (BSA) nor a low-affinity iron binding form of human serum albumin affected neurogenesis. For further information on the low-affinity iron binding form of human serum albumin (rHSA) see Silva et al., 2009. Biochimica et Biophysica Acta, Vol 1794, p1449-1458. Holo-transferrin (HoloTf), the iron-saturated form of transferrin, was also unable to induce differentiation of the SH-SY5Y cells.

Surprisingly, apo-ferritin, the iron-poor form of ferritin, another high-affinity iron binding protein with multiple iron binding sites, was ineffective at inducing differentiation of the SH-SY5Y cells. This furthered the hypothesis that iron binding is not the sole mechanism of action for the neurogenic potential of ApoTf. Unexpectedly, apo-lactoferrin also induced differentiation of these cells. Apo-lactoferrin is a structural and functional homologue of apo-transferrin but found in breast milk rather than plasma.

Apo-lactoferrin has 61% identity with apo-transferrin, whereas apo-ferritin and Human Serum Albumin (HSA) are structurally unrelated to either apo-transferrin or apo-lactoferrin.

### Example 5: ApoTf Induced Differentiation of SH-SY5Y cells is Not Through Hypoxia Inducible Factor 1α (HIF-1α)

It has been reported that both ApoTf and HoloTf can induce HIF-1α production leading to associated neuroprotective effects (US2016008437 to Grifols Worldwide Operations limited, the contents of which are incorporated herein by reference). While this is a beneficial attribute prior to death of a neuron, neuroprotection does not benefit the patient once a neuronal cell is dead. Neurogenesis, on the other-hand, benefits the patient after the insult because it can regenerate new neuronal cells.

In substantiation of the premises that ApoTf is mediating neurogenesis outside of the HIF pathway the present inventors tested a well-known, highly specific prolyl hydroxylase (PHD2) inhibitor in the SH-SY5Y cell differentiation assay. IOX2 (N-[[1,2-Dihydro-4-hydroxy-2-oxo-1-(phenylmethyl)-3-quinolinyl]carbonyl]glycine), a small molecule inhibitor of PHD2 is known to activate the HIF pathway through its actions on PHD2. See Chowdhury et al., 2013. ACS Chem. Biol. Vol 8, p1488. IOX2 has an IC₅₀ of 22 nM for inhibition of PHD2 and can induce up-regulation of HIF-1α in undifferentiated SH-SY5Y with concentrations as little as 1 μM (Ross, US2016008437 supra).

Undifferentiated SH-SY5Y cells were seeded and treated as described in Example 1. Neurite growth was assessed by imaging and image analysis. At the time of analysis, a 10× solution of Tubulin Tracker (Molecular Probes, T34075) and Hoechst 33342 (Molecular Probes #H3570) nuclear stain was prepared. Briefly, Tubulin Tracker dissolved in DMSO was diluted 1:1 with Pluronic F-127 and further diluted into HBSS to generate a 10× solution. Hoechst 33342 was added to the HBSS-Tubulin Tracker solution at 10 μg/mL to generate at 10× nuclear stain. The 10× staining solution (10 μL) was added directly to treated assay wells and incubated at 37° C. for 30 minutes. Following incubation, 110 μL of 0.4% Trypan Blue was added directly to assay wells and imaged on a Molecular Devices Nano imaging instrument. Nine images/well were acquired in the blue (Nuclei) and green (Tubulin) fluorescent channels for each image. After obtaining images, the MetaExpress Neurite Outgrowth analysis module (Molecular Devices) was used to identify cell bodies and quantify neurites. The total number of neurite branches were divided by the total number of cells imaged to account for different numbers of cells. The Outgrowth Fold Change was determined by setting the untreated control to a value of 1 with all other treatments shown relative to untreated control. ApoTf was obtained and purified from pooled human plasma and dosed at a final concentration of 0.2 mg/mL. IOX2 was obtained from Tocris (Cat #4451), resuspended and stored by the manufacturer's recommendations.

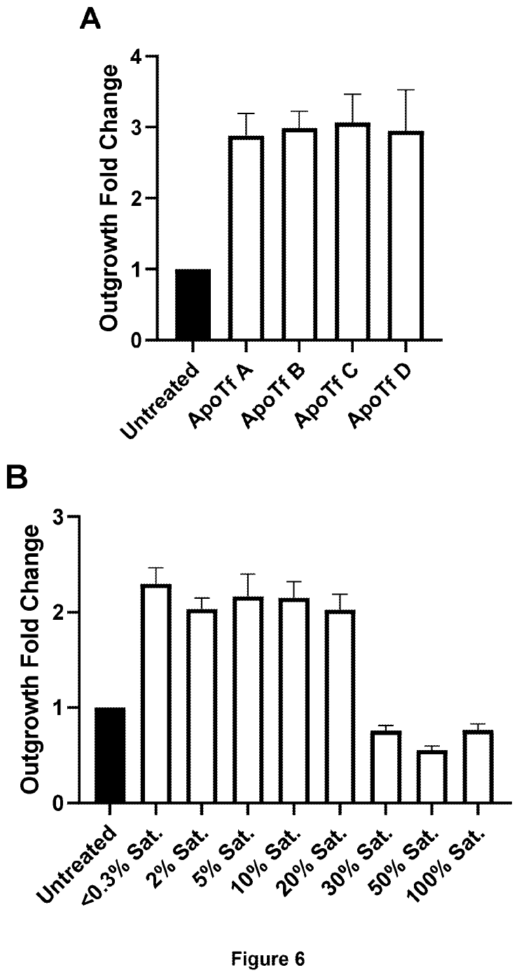

From FIG. 5 it is evident that no neurite outgrowth or differentiation was observed in the IOX2-treated cells. Even at very high concentrations of 4 μM IOX2 no effect was observable (4-fold higher than concentrations reported in US2016008437 to induce of HIF-1α in SH-SY5Y, and over 180-fold higher than the concentration that Chowdhury determined as the IC₅₀ for PHD2 proteins). These data, in combination with the lack of neurogenesis with HoloTf (Example 4), indicate that HIF-1α does not play a role in differentiating SH-SY5Y cells.

### Example 6: Role of Iron Saturation in Transferrin Efficacy

ApoTf, with various purities and iron saturation amounts, as outlined in Table 1 were assessed for their neurogenic potential. The transferrin samples were prepared according to the procedures/methodology known by those skilled in the art and detailed in section 21.4 of L von Bonsdorff, et al., Transferrin, Ch 21, pg 301-310, Production of Plasma Proteins for Therapeutic Use, Eds. J. Bertolini, et al., Wiley, 2013 [Print ISBN:9780470924310 |Online ISBN:9781118356807], the contents of which are incorporated herein by reference.

Protein purity was determined by SDS-PAGE. Iron saturation levels were determined using ICP-AES in accordance with the procedures outlined in Manley et al., J Biol Inorg Chem (2009) 14:61-74, the contents of which are incorporated herein by reference.

TABLE 1

| | Protein | Iron | |
|---|---|---|---|
| | Purity | Saturation | |
| Sample Name | (%) | (%) | Source |
| ApoTransferrin A | 99.11 | 0.27 | Grifols - prepared in house |
| ApoTransferrin B | 98.57 | 0.59 | Grifols - prepared in house |
| ApoTransferrin C | 96.72 | 0.24 | Grifols - prepared in house |
| ApoTransferrin D | 94.35 | Not | Athens Research & |
| | | Determined | Technology Inc., Cat# |
| | | | 16-16A32001-BPG |
| HoloTransferrin | 99.0 | 100 | Grifols - prepared in house |

Undifferentiated SH-SY5Y cells were treated as described in Example 1. Neurite growth was assessed by imaging and image analysis. At the time of analysis, a 10× solution of Tubulin Tracker (Molecular Probes, T34075) and Hoechst 33342 (Molecular Probes #H3570) nuclear stain was prepared. Briefly, Tubulin Tracker dissolved in DMSO was diluted 1:1 with Pluronic F-127 and further diluted into HBSS to generate a 10× solution. Hoechst 33342 was added to the HBSS-Tubulin Tracker solution at 10 μg/mL to generate at 10× nuclear stain. The 10× staining solution (10 μL) was added directly to treated assay wells and incubated at 37° C. for 30 minutes. Following incubation, 110 μL of 0.4% Trypan Blue was added directly to assay wells and imaged on a Molecular Devices Nano imaging instrument. Nine images/well were acquired in the blue (Nuclei) and green (Tubulin) fluorescent channels for each image. After obtaining images, the MetaExpress Neurite Outgrowth analysis module (Molecular Devices) was used to identify cell bodies and quantify neurites. The total number of neurite branches were divided by the total number of cells imaged to account for different numbers of cells. The Outgrowth Fold Change was determined by setting the untreated control to a value of 1 with all other treatments shown relative to untreated control.

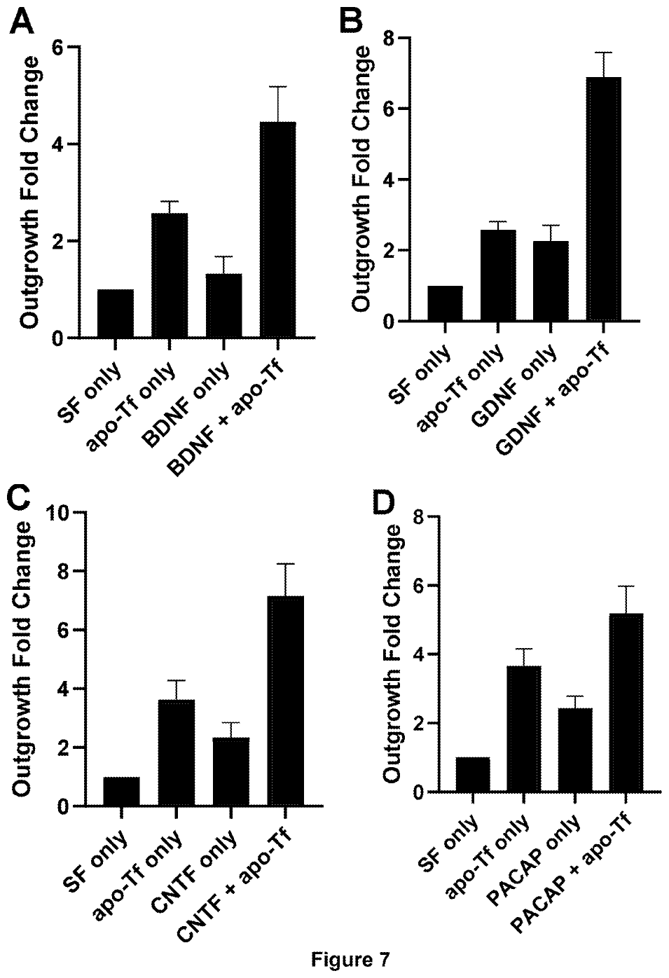

FIG. 6A plots the effect of ApoTf A-D, purity & iron content outlined in Table 1, dosed at a final concentration of 0.2 mg/mL on neurite outgrowth in SH-SY5Y cells. FIG. 6B plots transferrin with various iron saturation levels (listed on the X-axis) dosed at final concentrations of 0.2 mg/mL on neurite outgrowth in SH-SY5Y cells.

ApoTf (<0.3% Saturation) and, HoloTf (100% Saturation) were prepared after purification of transferrin from pooled human plasma as outlined in von Bonsdorff, vide supra. The various iron saturation contents were generated by mixing ApoTf and HoloTf to generate the indicated percent saturations plotted in FIG. 6B.

From FIG. 6A we see that all ApoTf preparations (ApoTf A-D), even the sample with a protein purity of only 94%, were able to induce neurogenic differentiation of SH-SY5Y. FIG. 6B illustrates effect the degree of iron saturation had on the ability of transferrin to induce differentiation of the SH-SY5Y cells. In this example, ApoTf or HoloTf with protein purities of at least 99% were mixed in various ratios to determine the effect of iron saturation/content. Transferrin with an iron saturation content less than 30% showed neurogenic potential.

### Example 7: Apo-Transferrin Acts Synergistically with Neurotrophic Protein and Peptide Factors to Induce Differentiation

Several neurotrophic protein factors have been considered for clinical use for stimulation of neurogenesis in neurodegenerative conditions and after traumatic brain injury. See Houlton et al., 2019. Frontiers in Neurosci., Vol. 13, Article 790; Weissmiller and Wu, 2012. Translational Neurodegeneration, Vol. 1:14; Apfel, 2001. Clin Chem Lab Med., Vol. 39(4), p351.

Proteins from three neurotrophic superfamilies were tested for function in combination with ApoTf. These neurotrophic proteins are: BDNF (brain-derived neurotrophic factor; NGF superfamily), GNDF (glial cell line-derived neurotrophic factor; TGF-β superfamily), and CNTF (cilliary neurotrophic factor-1; neurokine superfamily). In addition, another known neurotrophic peptide, PACAP (amino acids 1-38 of pituitary adenylate cyclase-activating polypeptide), was assessed for function in combination with ApoTf.

Undifferentiated SH-SY5Y cells were treated as described in Example 1. Neurite growth was assessed by imaging and image analysis. At the time of analysis, a 10× solution of Tubulin Tracker (Molecular Probes, T34075) and Hoechst 33342 (Molecular Probes #H3570) nuclear stain was prepared. Briefly, Tubulin Tracker dissolved in DMSO was diluted 1:1 with Pluronic F-127 and further diluted into HBSS to generate a 10× solution. Hoechst 33342 was added to the HBSS-Tubulin Tracker solution at 10 μg/mL to generate at 10× nuclear stain. The 10× staining solution (10 μL) was added directly to treated assay wells and incubated at 37° C. for 30 minutes. Following incubation, 110 μL of 0.4% Trypan Blue was added directly to assay wells and imaged on a Molecular Devices Nano imaging instrument. Nine images/well were acquired in the blue (Nuclei) and green (Tubulin) fluorescent channels for each image. After obtaining images, the MetaExpress Neurite Outgrowth analysis module (Molecular Devices) was used to identify cell bodies and quantify neurites. The total number of neurite branches were divided by the total number of cells imaged to account for different numbers of cells. The Outgrowth Fold Change was determined by setting the untreated control to a value of 1 with all other treatments shown relative to untreated control.

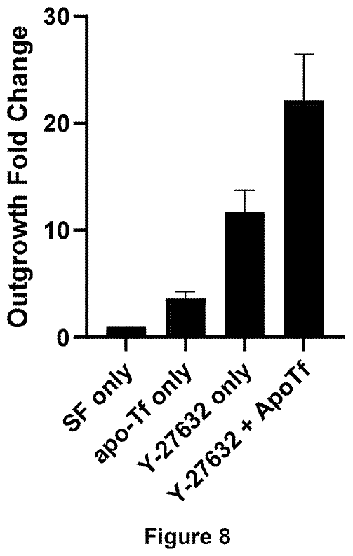

In FIGS. 7A-7D ApoTf was dosed at a final concentration of 0.1 mg/mL either alone or in combination with the indicated neurotrophic factor. (A) BDNF was obtained from Peprotech (Cat #450-02) and dosed at 25 ng/mL. (B) GDNF was obtained from Peprotech (Cat #450-10) and dosed at 1000 ng/mL. (C) CNTF was obtained from Peprotech (Cat #450-13) and dosed at 250 ng/mL. (D) PACAP was obtained from Tocris (Cat #1186) and dosed at 200 nM. The abbreviation SF denotes serum free media.

Reviewing each of FIGS. 7A-7D it is apparent that each of the neurotrophic factors, and the peptide fragment induced differentiation of SH-SY5Y cells to different degrees. In some cases, like BDNF, differentiation was not induced by the neurotrophic factor in the absence of ApoTf at the concentrations tested. In the all of the experiments presented, the neurotrophic factors combined with ApoTf induced greater differentiation than the molecules tested alone. Unexpectedly, ApoTf exhibits a synergistic effect with other neurotrophic factors and peptides on neurite outgrowth in SH-SY5Y cells.

### Example 8: Apo-Transferrin Acts Synergistically to Induce Differentiation with Neurogenic Small Molecules

The ability of ApoTf to act alongside non-protein based, neurogenic small molecule compounds was tested in Example 7. ApoTf was assessed in combination with the neurogenic compound Y-27632 [trans-4-[(1R)-1-Aminoethyl]-N-4-pyridinylcyclohexanecarboxamide dihydrochloride]. Y-27632 is a Rock1 and Rock2 (Rho kinase) inhibitor. Inhibition of Rock1 and 2 by small molecules has the known ability to induce neuronal differentiation, including SH-SY5Y cells. See Dyberg et al., 2017. PNAS Vol 114 (32), E6603-E6612.

Undifferentiated SH-SY5Y cells were treated as described in Example 1. Neurite growth was assessed by imaging and image analysis. At the time of analysis, a 10× solution of Tubulin Tracker (Molecular Probes, T34075) and Hoechst 33342 (Molecular Probes #H3570) nuclear stain was prepared. Briefly, Tubulin Tracker dissolved in DMSO was diluted 1:1 with Pluronic F-127 and further diluted into HBSS to generate a 10× solution. Hoechst 33342 was added to the HBSS-Tubulin Tracker solution at 10 μg/mL to generate at 10× nuclear stain. The 10× staining solution (10 μL) was added directly to treated assay wells and incubated at 37° C. for 30 minutes. Following incubation, 110 μL of 0.4% Trypan Blue was added directly to assay wells and imaged on a Molecular Devices Nano imaging instrument. Nine images/well were acquired in the blue (Nuclei) and green (Tubulin) fluorescent channels for each image. After obtaining images, the MetaExpress Neurite Outgrowth analysis module (Molecular Devices) was used to identify cell bodies and quantify neurites. The total number of neurite branches were divided by the total number of cells imaged to account for different numbers of cells. The Outgrowth Fold Change was determined by setting the untreated control to a value of 1 with all other treatments shown relative to untreated control. ApoTf was dosed at a final concentration of 0.1 mg/mL either alone or in combination with the indicated small molecule. Y-27632 was obtained from Tocris (Cat #1254) and dosed at 50 μM.

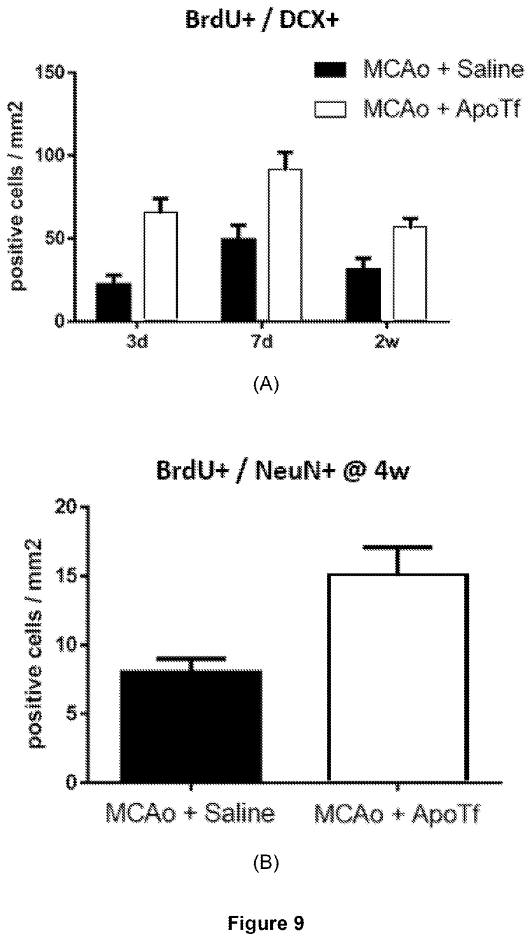

FIG. 8 illustrates that Y-27632 itself is a strongly neurogenic compound, however, in the presence of ApoTf, the neurogenic effect was synergistic showing an effect beyond that exhibited by either molecule alone. The ability of ApoTf to act synergistically with a number of known protein, peptide, and small molecule neurogenic entities is an unexpected and surprising intriguing finding.

### Example 9: Apo-Transferrin Promotes New Neuroblast and Mature Neuron Formation in Brains of Animals with Transient MCAo

C57BL/6J mice (ca. 20 g) were anaesthetized under isoflurane and, after dissection, a 6.0 silicon-coated monofilament suture was inserted into the external carotid artery to occlude the middle cerebral artery (MCAo). The occlusion was carried out for 60 minutes under temperature control. Within two hours after removing the occlusion, animals were assessed on a 7-point ‘neuroscore’ scale to identify candidates with a visual demonstration of stroke on a scale from 0 (no observable deficit) to 6 (moribund)—the extension of the contralateral forepaw, the severity of circling, the loss of walking and consciousness were all taken into consideration.
0=no observable deficit
1=failure to extend the contralateral forepaw
2=mild circling behavior when picked up by the tail, <50% attempts to rotate to the contralateral side
3=mild consistent circling, >50% attempts to rotate to the contralateral side
4=consistent and strong circling, the mouse holds a rotation position for more than 1 to 2 seconds, with its nose almost reaching its tail
5=severe rotation with falling in a direction contralateral to the infarct, loss of walking or righting reflex, and
6=depressed level of consciousness, comatose, or moribund

Only animals with a ‘neuroscore’ of 4 or greater were considered for further testing (n=8-10 animals/group). At six hours post-occlusion, mice were injected daily with 350 mg/kg of Apo-transferrin (i.p.) or an equal volume of saline for a total of seven days, as well as with 50 mg/kg Bromodeoxyuridine (BrdU; Sigma-Aldrich Chemical, St. Louis, Mo.), both articles delivered by intraperitoneal administration. Weight changes were monitored daily.

At the times indicated in Example 9, brains were prepared for analysis by transcardial perfusion with ice cold heparinized saline (heparin at 2.5 IU/ml) in order to remove blood from the brains. Fresh brains were removed leaving the right cerebellum attached to whole left-brain hemisphere to help orientate the brain block in sectioning. The whole left hemisphere block was placed in 4% paraformaldehyde in 0.1 M phosphate buffer (PB) for 24 hours at +4° C., then cryoprotected in 30% sucrose in 0.1 M PB for 2-3 days on a shaker at +4° C. The brain was then blotted to remove excess liquid, placed on top of a vial cork and frozen on top of liquid nitrogen; the block was then stored at −80° C. until cryostat sections are obtained. Neurogenesis was evaluated by immunohistochemistry using antibodies against BrdU and Doublecortin (DCX) to quantify new neuroblasts or against BrdU and NeuN to quantify new neurons in the dentate gyrus of the brain.

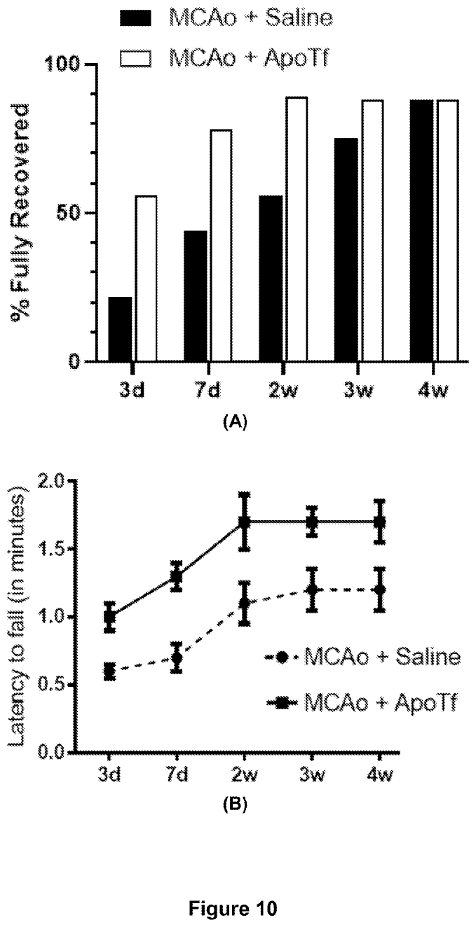

FIG. 9A demonstrates that administration of ApoTf increases the number of neuroblasts over a two-week period, while FIG. 9B shows that after 4 weeks the number of newly formed mature neurons are higher in ApoTf-treated mice. These data suggest that the neuroblasts created early in neurogenesis may go on to differentiate further into new mature neurons. These results therefore suggest that apo-transferrin can promote aspects of neurogenesis above and beyond the neurogenesis that is normally induced in response to ischemic stroke.

### Example 10: Apo-Transferrin Promotes Recovery, Motor Skills, and Cognition in a Mouse Model of Transient MCAo Stroke

Mice were prepared as indicated in Example 9 above and were subjected to assessments at 3 days and 1, 2, 3, 4 weeks post-MCAo, as indicated (n=8-10 animals/group). Motor coordination (i.e. equilibrium behavior and locomotor ability as a function of cortico-striatal function) was assessed using a rotarod test. Learning and memory capacity were measured using the NORT (novel object recognition test; for example, Anglada-Huguet et al., 2014, Molecular Neurobiology, vol. 49, pages 784-795; Denninger et al., 2018, J. Vis. Exp., vol. 141, e58593).

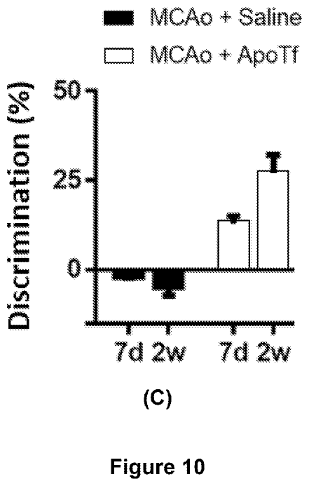

FIG. 10A shows that ApoTf significantly improves the rate at which the animals recover after MCA occlusion (MCAo) over the course of 3-14 days, and, possibly, longer. As described in Example 9, all animals in the study have an initial neuroscore of 4 or higher when assessed 2 hr after the MCAo. The percentage of animals that have no observable deficit (i.e. a neuroscore of ‘0’, as described in Example 9) are shown as a function of time after MCAo, as indicated on the x-axis.

FIG. 10B shows that animals treated with apo-transferrin have increased motor and balance skills after MCAo. The time that it takes an animal to fall (Latency to fall) from the rotarod is shown vs time after MCAo. The motor skills of the mice reflect those of the neuroscore in FIG. 10A, with an increased rate of improvement in motor/balance skills, as measured by the amount of time animals stayed on the rotarod apparatus. While the recovery as measured by neuroscore is the same in both groups by 4 weeks, the mice treated with apoTf have better overall abilities to remain on the rotarod. FIG. 10C provides evidence that administration of ApoTf increases learning recognition functions over at least a two-week period following administration of protein. The Discrimination (%) is a description of an animal's memory, as measured by the percentage of time in which an animal investigates newly presented objects compared to the time they investigate previously presented objects. Animals with better cognition and memory spend more time with the novel object due to memories of the object previously presented to the animal; this is shown as increased % Discrimination. The animals treated with apoTf have a higher ‘percent Discrimination’ as compared to saline-treated mice, suggesting that after MCAo, the apoTf-treated mice recover their cognitive abilities better.

Taken together, the data from FIGS. 10A to 10C suggest the neurogenesis promoted by apoTf translates to better motor ability and cognition within subjects.

### Sequences

The sequences referred to in the preceding text are outlined below in fasta format.

Human Transferrin [UniProt Q06AH7]
protein sequence
SEQ ID NO: 1
VPDKTVRWCAVSEHEATKCQSFRDHMKSVIPSDGP
SVACVKKASYLDCIRAIAANEADAVTLDAGLVYDA
YLAPNNLKPVVAEFYGSKEDPQTFYYAVAVVKKDS
GFQMNQLRGKKSCHTGLGRSAGWNIPIGLLYCDLP
EPRKPLEKAVANFFSGSCAPCADGTDFPQLCQLCP
GCGCSTLNQYFGYSGAFKCLKDGAGDVAFVKHSTI
FENLANKADRDQYELLCLDNTRKPVDEYKDCHLAQ
VPSHTVVARSMGGKEDLIWELLNQAQEHFGKDKSK
EFQLFSSPHGKDLLFKDSAHGFLKVPPRMDAKMYL
GYEYVTAIRNLREGTCPEAPTDECKPVKWCALSHH
ERLKCDEWSVNSVGKIECVSAETTEDCIAKIMNGE
ADAMSLDGGFVYIAGKCGLVPVLAENYNKSDNCED
TPEAGYFAVAVVKKSASDLTWDNLKGKKSCHTAVG
RTAGWNIPMGLLYNKINHCRFDEFFSEGCAPGSKK
DSSLCKLCMGSGLNLCEPNNKEGYYGYTGAFRCLV
EKGDVAFVKHQTVPQNTGGKNPDPWAKNLNEKDYE
LLCLDGTRKPVEEYANCHLARAPNHAVVTRKDKEA
CVHKILRQQQHLFGSNVTDCSGNFCLFRSETKDLL
FRDDTVCLAKLHDRNTYEKYLGEEYVKAVGNLRKC
STSSLLEACTFRRP
Human Lactoferrin [UniProt P02788]
protein sequence
SEQ ID NO: 2
GRRRRSVQWCTVSQPEATKCFQWQRNMRRVRGPPV
SCIKRDSPIQCIQAIAENRADAVTLDGGFIYEAGL
APYKLRPVAAEVYGTERQPRTHYYAVAVVKKGGSF
QLNELQGLKSCHTGLRRTAGWNVPIGTLRPFLNWT
GPPEPIEAAVARFFSASCVPGADKGQFPNLCRLCA
GTGENKCAFSSQEPYFSYSGAFKCLRDGAGDVAFI
RESTVFEDLSDEAERDEYELLCPDNTRKPVDKFKD
CHLARVPSHAVVARSVNGKEDAIWNLLRQAQEKFG
KDKSPKFQLFGSPSGQKDLLFKDSAIGFSRVPPRI
DSGLYLGSGYFTAIQNLRKSEEEVAARRARVVWCA
VGEQELRKCNQWSGLSEGSVTCSSASTTEDCIALV
LKGEADAMSLDGGYVYTAGKCGLVPVLAENYKSQQ
SSDPDPNCVDRPVEGYLAVAVVRRSDTSLTWNSVK
GKKSCHTAVDRTAGWNIPMGLLFNQTGSCKFDEYF
SQSCAPGSDPRSNLCALCIGDEQGENKCVPNSNER
YYGYTGAFRCLAENAGDVAFVKDVTVLQNTDGNNN
DAWAKDLKLADFALLCLDGKRKPVTEARSCHLAMA
PNHAVVSRMDKVERLKQVLLHQQAKFGRNGSDCPD
KFCLFQSETKNLLFNDNTECLARLHGKTTYEKYLG
PQYVAGITNLKKCSTSPLLEACEFLRK
Y188F Transferrin N-lobe mutant
protein
SEQ ID NO: 3
VPDKTVRWCAVSEHEATKCQSFRDHMKSVIPSDGP
SVACVKKASYLDCIRAIAANEADAVTLDAGLVYDA
YLAPNNLKPVVAEFYGSKEDPQTFYYAVAVVKKDS
GFQMNQLRGKKSCHTGLGRSAGWNIPIGLLYCDLP
EPRKPLEKAVANFFSGSCAPCADGTDFPQLCQLCP
GCGCSTLNQYFGFSGAFKCLKDGAGDVAFVKHSTI
FENLANKADRDQYELLCLDNTRKPVDEYKDCHLAQ
VPSHTVVARSMGGKEDLIWELLNQAQEHFGKDKSK
EFQLFSSPHGKDLLFKDSAHGFLKVPPRMDAKMYL
GYEYVTAIRNLREGTCPEAPTDECKPVKWCALSHH
ERLKCDEWSVNSVGKIECVSAETTEDCIAKIMNGE
ADAMSLDGGFVYIAGKCGLVPVLAENYNKSDNCED
TPEAGYFAVAVVKKSASDLTWDNLKGKKSCHTAVG
RTAGWNIPMGLLYNKINHCRFDEFFSEGCAPGSKK
DSSLCKLCMGSGLNLCEPNNKEGYYGYTGAFRCLV
EKGDVAFVKHQTVPQNTGGKNPDPWAKNLNEKDYE
LLCLDGTRKPVEEYANCHLARAPNHAVVTRKDKEA
CVHKILRQQQHLFGSNVTDCSGNFCLFRSETKDLL
FRDDTVCLAKLHDRNTYEKYLGEEYVKAVGNLRKC
STSSLLEACTFRRP
Y95F/Y188F Transferrin N-lobe
mutant protein
SEQ ID 4
VPDKTVRWCAVSEHEATKCQSFRDHMKSVIPSDGP
SVACVKKASYLDCIRAIAANEADAVTLDAGLVYDA
YLAPNNLKPVVAEFYGSKEDPQTFFYAVAVVKKDS
GFQMNQLRGKKSCHTGLGRSAGWNIPIGLLYCDLP
EPRKPLEKAVANFFSGSCAPCADGTDFPQLCQLCP
GCGCSTLNQYFGFSGAFKCLKDGAGDVAFVKHSTI
FENLANKADRDQYELLCLDNTRKPVDEYKDCHLAQ
VPSHTVVARSMGGKEDLIWELLNQAQEHFGKDKSK
EFQLFSSPHGKDLLFKDSAHGFLKVPPRMDAKMYL
GYEYVTAIRNLREGTCPEAPTDECKPVKWCALSHH
ERLKCDEWSVNSVGKIECVSAETTEDCIAKIMNGE
ADAMSLDGGFVYIAGKCGLVPVLAENYNKSDNCED
TPEAGYFAVAVVKKSASDLTWDNLKGKKSCHTAVG
RTAGWNIPMGLLYNKINHCRFDEFFSEGCAPGSKK
DSSLCKLCMGSGLNLCEPNNKEGYYGYTGAFRCLV
EKGDVAFVKHQTVPQNTGGKNPDPWAKNLNEKDYE
LLCLDGTRKPVEEYANCHLARAPNHAVVTRKDKEA
CVHKILRQQQHLFGSNVTDCSGNFCLFRSETKDLL
FRDDTVCLAKLHDRNTYEKYLGEEYVKAVGNLRKC
STSSLLEACTFRRP
Y426F/Y517F Transferrin C-lobe
mutant protein
SEQ ID NO: 5
VPDKTVRWCAVSEHEATKCQSFRDHMKSVIPSDGP
SVACVKKASYLDCIRAIAANEADAVTLDAGLVYDA
YLAPNNLKPVVAEFYGSKEDPQTFYYAVAVVKKDS
GFQMNQLRGKKSCHTGLGRSAGWNIPIGLLYCDLP
EPRKPLEKAVANFFSGSCAPCADGTDFPQLCQLCP
GCGCSTLNQYFGYSGAFKCLKDGAGDVAFVKHSTI
FENLANKADRDQYELLCLDNTRKPVDEYKDCHLAQ
VPSHTVVARSMGGKEDLIWELLNQAQEHFGKDKSK
EFQLFSSPHGKDLLFKDSAHGFLKVPPRMDAKMYL
GYEYVTAIRNLREGTCPEAPTDECKPVKWCALSHH
ERLKCDEWSVNSVGKIECVSAETTEDCIAKIMNGE
ADAMSLDGGFVYIAGKCGLVPVLAENYNKSDNCED
TPEAGFFAVAVVKKSASDLTWDNLKGKKSCHTAVG
RTAGWNIPMGLLYNKINHCRFDEFFSEGCAPGSKK
DSSLCKLCMGSGLNLCEPNNKEGYYGFTGAFRCLV
EKGDVAFVKHQTVPQNTGGKNPDPWAKNLNEKDYE
LLCLDGTRKPVEEYANCHLARAPNHAVVTRKDKEA
CVHKILRQQQHLFGSNVTDCSGNFCLFRSETKDLL
FRDDTVCLAKLHDRNTYEKYLGEEYVKAVGNLRKC
STSSLLEACTFRRP
BDNF
SEQ ID NO: 6
MFHQVRRVMTILFLTMVISYFGCMKAAPMKEANIR
GQGGLAYPGVRTHGTLESVNGPKAGSRGLTSLADT
FEHVIEELLDEDQKVRPNEENNKDADLYTSRVMLS
SQVPLEPPLLFLLEEYKNYLDAANMSMRVRRHSDP
ARRGELSVCDSISEWVTAADKKTAVDMSGGTVTVL
EKVPVSKGQLKQYFYETKCNPMGYTKEGCRGIDKR
HWNSQCRTTQSYVRALTMDS KKRIGWRFIRIDTS
CVCTLT IKRGR
GDNF
SEQ ID NO: 7
MQSLPNSNGAAAGRDFKMKLWDVVAVCLVLLHTAS
AFPLPAANMPEDYPDQFDDVMDFIQATIKRLKRSP
DKQMAVLPRRERNRQAAAANPENSRGKGRRGQRGK
NRGCVLTAIHLNVTDLGLGYETKEELIFRYCSGSC
DAAETTYDKILKNLSRNRRLVSDKVGQACCRPIAF
DDDLSFLDDNLVYHILRKHSAKRCGCI
CNTF
SEQ ID NO: 8
MAFTEHSPLTPHRRDLCSRSIWLARKIRSDLTALT
ESYVKHQGLNKNINLDSADGMPVASTDOWSELTEA
ERLQENLQAYRTFHVLLARLLEDQQVHFTPTEGDF
HQAIHTLLLQVAAFAYQIEELMILLEYKIPRNEAD
GMPINVGDGGLFEKKLWGLKVLQELSQWTVRSIHD
LRFISSHQTGIPARGSHYIANNKKM
PACAP
SEQ ID NO: 9
HSDGIFTDSYSRYRKQMAVKKYLAAVLGKRYKQRV
KNK

1. A method of promoting and/or inducing generation of new neural cells in a patient that has suffered a neurodegenerative event arising from at least one of a traumatic brain injury, a non-traumatic brain injury, a spinal cord injury, a peripheral nerve injury, or peripheral neuropathy,
the method comprising administering a therapeutically effective amount of a protein selected from transferrin, lactoferrin, and combinations thereof to the patient in need thereof.

2. The method of claim 1, wherein the therapeutically effective amount of transferrin or lactoferrin administered to the patient has an iron saturation of less than about 20%.

3. The method of claim 1, wherein the protein is human transferrin.

4. The method of claim 1, wherein the transferrin is plasma-derived or recombinant.

5. The method of claim 4, wherein the recombinant transferrin is a mutant transferrin selected from the group consisting of:
i) Y188F mutant comprising the amino acid sequence set forth in SEQ ID NO: 3;
ii) Y95F/Y188F mutant comprising the amino acid sequence set forth in SEQ ID NO: 4;
iii) Y426F/Y517F mutant comprising the amino acid sequence set forth in SEQ ID NO: 5; and
iv) combinations thereof.

6. The method of claim 1, wherein the transferrin is a domain of a fusion protein, and the fusion partner is an immunoglobulin Fc domain.

7. The method of claim 1, wherein the traumatic brain injury or spinal cord injury is caused by at least one of road a traffic accident, an assault, a sporting collision, or an unprotected fall.

8. The method of claim 1, wherein the non-traumatic brain injury is caused by at least one of an ischaemic stroke, a haemorrhagic stroke, cerebral hypoxia, cerebral anoxia, consumption of chemical toxins, hydrocephalus, meningitis, or encephalitis.

9. The method of claim 1, wherein the neurodegenerative event arises from a stroke selected from the group consisting of a ischemic stroke and a haemorrhagic stroke.

10. The method of claim 1, further comprising administering a serum or plasma protein selected from the group consisting of Albumin, Alpha-1 Antitrypsin/Alpha-1 Proteinase Inhibitor, Antithrombin, polyclonal immunoglobulins, polyspecific immunoglobulins, C1 esterase inhibitor, Transthyretin, and combinations thereof to the patient in addition to the protein selected from transferrin, lactoferrin, and combinations thereof.

11-21. (canceled)

22. A method of stimulating neural cell development in a patient that has suffered a neurodegenerative event arising from at least one of a traumatic brain injury, a non-traumatic brain injury, a spinal cord injury, a peripheral nerve injury, or peripheral neuropathy,
the method comprising administering a therapeutically effective amount of a protein selected from transferrin, lactoferrin, and combinations thereof to the patient in need thereof.

23. The method of claim 2Z wherein the therapeutically effective amount of transferrin or lactoferrin administered to the patient has an iron saturation of less than about 20%.

24. The method of claim 22, wherein the protein is human transferrin.

25. The method of claim 22, wherein the transferrin is plasma derived or recombinant.

26. The method of claim 25, wherein the recombinant transferrin is a mutant transferrin selected from the group consisting of:
v) Y188F mutant comprising the amino acid sequence set forth in SEQ ID NO: 3;
vi) Y95F/Y188F mutant comprising the amino acid sequence set forth in SEQ ID NO: 4;
vii) Y426F/Y517F mutant comprising the amino acid sequence set forth in SEQ ID NO: 5; and
viii) combinations thereof.

27. The method of claim 22, wherein the transferrin is a domain of a fusion protein, and the fusion partner is an immunoglobulin Fc domain.

28. The method of claim 22, wherein the traumatic brain injury or spinal cord injury is caused by at least one of road a traffic accident, an assault, a sporting collision, or an unprotected fall.

29. The method of claim 22, wherein the non-traumatic brain injury is caused by at least one of an ischaemic stroke, a haemorrhagic stroke, cerebral hypoxia, cerebral anoxia, consumption of chemical toxins, hydrocephalus, meningitis, or encephalitis.

30. The method of claim 22, wherein the neurodegenerative event arises from a stroke selected from the group consisting of a ischemic stroke and a haemorrhagic stroke.

31. The method of claim 22, further comprising administering a serum or plasma protein selected from the group consisting of Albumin, Alpha-1 Antitrypsin/Alpha-1 Proteinase Inhibitor, Antithrombin, polyclonal immunoglobulins, polyspecific immunoglobulins, C1 esterase inhibitor, Transthyretin, and combinations thereof to the patient in addition to the protein selected from transferrin, lactoferrin, and combinations thereof.

32-42. (canceled)


SEQUENCE LISTING (9 sequences)

SEQ ID NO: 1 - protein, 679 aa, Homo sapiens
VPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 2 - protein, 692 aa, Homo sapiens
GRRRRSVQWCTVSQPEATKCFQWQRNMRRVRGPPVSCIKRDSPIQCIQAIAENRADAVTLDGGFIYEAGLAPYKLRPVAAEVYGTERQPRTHYYAVAVVKKGGSFQLNELQGLKSCHTGLRRTAGWNVPIGTLRPFLNWTGPPEPIEAAVARFFSASCVPGADKGQFPNLCRLCAGTGENKCAFSSQEPYFSYSGAFKCLRDGAGDVAFIRESTVFEDLSDEAERDEYELLCPDNTRKPVDKFKDCHLARVPSHAVVARSVNGKEDAIWNLLRQAQEKFGKDKSPKFQLFGSPSGQKDLLFKDSAIGFSRVPPRIDSGLYLGSGYFTAIQNLRKSEEEVAARRARVVWCAVGEQELRKCNQWSGLSEGSVTCSSASTTEDCIALVLKGEADAMSLDGGYVYTAGKCGLVPVLAENYKSQQSSDPDPNCVDRPVEGYLAVAVVRRSDTSLTWNSVKGKKSCHTAVDRTAGWNIPMGLLFNQTGSCKFDEYFSQSCAPGSDPRSNLCALCIGDEQGENKCVPNSNERYYGYTGAFRCLAENAGDVAFVKDVTVLQNTDGNNNDAWAKDLKLADFALLCLDGKRKPVTEARSCHLAMAPNHAVVSRMDKVERLKQVLLHQQAKFGRNGSDCPDKFCLFQSETKNLLFNDNTECLARLHGKTTYEKYLGPQYVAGITNLKKCSTSPLLEACEFLRK

SEQ ID NO: 3 - protein, 679 aa, Artificial Sequence
  Y188F mutant
VPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGFSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 4 - protein, 679 aa, Artificial Sequence
  Y95F/Y188F mutant
VPDKTVRWCAVSEHEATKCQSFRDHMKSVIPSDGPSVACVKKASYLDCIRAIAANEADAVTLDAGLVYDAYLAPNNLKPVVAEFYGSKEDPQTFFYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGFSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 5 - protein, 679 aa, Artificial Sequence
  Y426F/Y517F mutant
VPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGFFAVAVVKKSASDLTWDNLKGKKSCHTAVGRTAGWNIPMGLLYNKINHCRFDEFFSEGCAPGSKKDSSLCKLCMGSGLNLCEPNNKEGYYGFTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 6 - protein, 255 aa, Homo sapiens
MFHQVRRVMTILFLTMVISYFGCMKAAPMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 7 - protein, 202 aa, Homo sapiens
MQSLPNSNGAAAGRDFKMKLWDVVAVCLVLLHTASAFPLPAANMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 8 - protein, 200 aa, Homo sapiens
MAFTEHSPLTPHRRDLCSRSIWLARKIRSDLTALTESYVKHQGLNKNINLDSADGMPVASTDQWSELTEAERLQENLQAYRTFHVLLARLLEDQQVHFTPTEGDFHQAIHTLLLQVAAFAYQIEELMILLEYKIPRNEADGMPINVGDGGLFEKKLWGLKVLQELSQWTVRSIHDLRFISSHQTGIPARGSHYIANNKKM

SEQ ID NO: 9 - protein, 38 aa, Homo sapiens
HSDGIFTDSYSRYRKQMAVKKYLAAVLGKRYKQRVKNK